(19) United States
(12) Patent Application Publication
(10) Pub. No.: US 2025/0108054 A1
(43) Pub. Date: Apr. 3, 2025

(54) METHODS FOR TREATING PERSISTENT OR CHRONIC IMMUNE THROMBOCYTOPENIA IN CHILDREN, ADOLESCENTS AND ADULTS BY ADMINISTERING (R)-2-[3-[4-AMINO-3-(2-FLUORO-4-PHENOXY-PHENYL)PYRAZOLO[3,4-D]PYRIMIDIN-1-YL]PIPERIDINE-1-CARBONYL]-4-METHYL-4-[4-(OXETAN-3-YL)PIPERAZIN-1-YL]PENT-2-ENENITRILE

(71) Applicant: Principia Biopharma Inc., Bridgewater, NJ (US)

(72) Inventors: Ahmed Daak, Concord, MA (US); Michelle Lee, Princeton Junction, NJ (US); Hailing Guo, Hillsborough, NJ (US)

(73) Assignee: Principia Biopharma Inc., Bridgewater, NJ (US)

(21) Appl. No.: 18/756,019

(22) Filed: Jun. 27, 2024

Related U.S. Application Data
(60) Provisional application No. 63/637,286, filed on Apr. 22, 2024.
(60) Provisional application No. 63/607,818, filed on Dec. 8, 2023.
(60) Provisional application No. 63/535,183, filed on Aug. 29, 2023.
(60) Provisional application No. 63/524,530, filed on Jun. 30, 2023.

(51) Int. Cl. A61K 31/519; A61K 45/06; A61P 7/00

(52) U.S. Cl. CPC A61K 31/519; A61K 45/06; A61P 7/00

(57) ABSTRACT

Methods for treating children, adolescents and adults with persistent or chronic immune thrombocytopenia comprising administering at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof are disclosed.

Disclosed herein are methods for treating immune thrombocytopenia. BTK inhibitors and pharmaceutical compositions comprising the same are also disclosed.

Immune thrombocytopenia, commonly referred to as ITP, is a rare acquired autoimmune disease with an estimated global prevalence of 10-23 per 100,000 people and an incidence of approximately 2-4 per 100,000 person-years in the general population, including both adult patients and patients under 18 years of age (Abrahamson et al. 2009, Christiansen et al. 2019, Feudjo-Tepie et al. 2008, Schoonen et al. 2009, Segal J B and Powe N R 2006, Terrell D R et al. 2010, Yong et al. 2010). The disease, which is characterized by the autoantibody-mediated destruction of platelets and their progenitor cells, has a heterogeneous pathophysiology that includes pathogenic immunoglobulin G (IgG) autoantibodies targeting antigens on the surface of platelets and their progenitor cells (e.g., glycoproteins aIIb/03 (GPIIb/IIIA), GPIa/IIa, and GPIb-IX-V) (Al-Samkari et al. 2020, Grodzielski et al. 2018, Zufferey et al. 2017). Autoantibody binding triggers platelet destruction and impaired platelet production through a number of mechanisms, including: antibody-coated cell phagocytosis through binding of autoantibodies to Fcγ receptors on macrophages; platelet clearance by C-type lectin receptor (CLEC4F) on hepatic Kupffer cells (e.g., Ashwell-Morell receptors); platelet lysis by the membrane attack complex; phagocytosis due to classical complement pathway activation; T cell-mediated cytotoxicity; and impaired megakaryocyte viability (Grodzielski et al. 2018, Peerschke et al. 2010, Reis et al. 2019, Zufferey et al. 2017).

The destruction of mature platelets and the impairment of platelet production results in thrombocytopenia (a platelet count below 100×10⁹/μL), putting patients at a high risk for bleeding, excessive bruising, and fatigue, as well as for life threatening intracranial bleeding. This predisposition to bleeding and thrombosis has an adverse impact on patient quality of life (QOL) (Adelborg et al. 2019, Efficace et al. 2016, Rodeghiero et al. 2009).

The pathophysiology of persistent and chronic ITP is similar in adolescents and adults, leading to similar clinical presentation and disease progression in both populations. Such similarities include presenting platelet counts, incidence and type of bleeding when platelet counts are <20×10⁹/μL, family history of thrombocytopenia (2% in children and 3% in adults), rates of treatment (80% of children and 71% of adults at presentation and 58% of children and adults at 6 months, with similarly decreasing rates within both groups at 12 and 24 months) (Kühne 2001; Schifferli 2018) and similar late remission rates among children and adults with persistent and chronic ITP at 12 and 24 months (Schifferli 2018).

Although there are differences between the adolescents and young children in terms of the etiology, pathophysiology, and clinical course, the currently available evidence shows that the disease characteristics in pediatric participants between 10 and less than 12 years of age is comparable to children above 12 years of age and adult population.

Current therapies for adults with ITP include initial treatment with intravenous immunoglobulin (IVIG) and corticosteroids (CS), and subsequent treatment with splenectomy, thrombopoietin receptor agonists (TPO-RAs), rituximab, fostamatinib, and immunosuppressive therapies (such as, e.g., mycophenolate mofetil (MMF) and cyclosporine). In general, pharmacotherapy (e.g., CS, IVIG, or anti-D immunoglobulin therapy) is used for symptomatic patients with low platelet counts for reducing platelet destruction and/or stimulating platelet production, with the goal of preventing bleeding (Cooper N and Ghanima W 2019, Kuter D J 2022).

The standard therapy for adult patients with newly diagnosed ITP consists of treatment with CS such as high-dose dexamethasone or oral prednisone/prednisolone, the prolonged use of which is associated with significant toxicity and should be avoided due to the potential for adverse events (Cooper N and Ghanima W 2019, Neunert et al. 2019). While most patients respond initially to CS, responses are typically not durable, and the rate of continued remission is low (Cooper N and Ghanima W 2019). Other first line therapies include IVIG and anti-D immunoglobulin.

Second line therapies for ITP include rituximab, TPO-RAs, fostamatinib, and splenectomy. Rituximab and TPO-RAs have shown durable on-treatment response rates of 60-80%, while fostamatinib has a durable response rate of ˜18% in a population of heavily pretreated ITP patients (Neunert et al. 2019, Mingot-Castellano et al. 2022, Singh et al. 2021). While splenectomy has the benefit of high response rates and durable off-treatment remission rates of 60-70%, it may be associated with short-term surgical complications as well as a long-term increased risk of thrombosis and infection (Mingot-Castellano et al. 2022, Singh et al. 2021). Additionally, thrombopoietin (TPO) mimetics (Bussel 2007) are approved for the treatment of patients with chronic ITP who have not had sufficient responses to CS, IVIG, or splenectomy.

ITP patients requiring ongoing treatment and patients who are unresponsive to current therapy have elevated mortality rates relative to the general population (Frederiksen et al. 2014, Norgaard et al. 2011). Adult patients with chronic thrombocytopenia have up to a 10% risk of bleeding/hemorrhage that increases with age, and intracranial hemorrhage has been reported in ˜1-2% of patients (Adelborg et al. 2019, Neunert et al. 2015, Norgaard et al. 2011). Besides the risk of bleeding, ITP patients may experience significant fatigue, cognitive impairment, fear of bleeding, and a negative impact on social and work activities, further attesting to the presence of a significant unmet need in this patient population (Frith et al. 2012, Terrell et al. 2020, Trotter P and Hill Q A 2018).

Novel, safe, and effective oral treatments to maintain platelet counts and improve outcomes in ITP patients would represent a significant therapeutic advantage over current standard of care. By way of non-limiting example, unmet needs in chronic and persistent ITP include: improving remission rates and durability; avoiding rapid increase of platelet counts/thrombosis risk; steroid-free regimens; and a tolerable and safe therapy that ensures good patient QOL. Thus, there is a need for novel oral therapies for treating ITP, including chronic and persistent ITP, that address some or all of these limitations of existing therapeutic modalities. Additionally, given the heterogeneity of mechanisms underlying the development of ITP, a single therapy or combination of therapies that can target multiple disease-associated pathways may be necessary to induce a sufficient and durable platelet response.

Bruton's agammaglobulinemia tyrosine kinase (BTK), an essential signaling element in B cells and innate immune cells, is expressed downstream of the B cell receptor (BCR), Fc-gamma receptor (FcγR), and Fc-epsilon receptor (FcgR) and serves a direct regulator of the NLRP3 inflammasome. BTK is a non-receptor tyrosine kinase and a member of the TEC family of kinases. BTK is essential to B cell differentiation, development, and antibody production. Illustratively, inhibition of BTK activity produces phenotypic changes consistent with blockade of the BCR, including the down-regulation of cell proliferation, differentiation, maturation, and survival, as well as the up-regulation of apoptosis.

Rather than acting in an “on/off switch” manner, BTK may be best viewed as an immune function “modulator” (Crofford L J et al., 2016; Pal Singh S et al., 2018). Important insights into BTK function come from loss of function analyses in humans and mice. Individuals with loss of function mutations in the BTK gene develop X-linked agammaglobulinemia (XLA), characterized by a complete absence of circulating B cells and plasma cells, and very low levels of immunoglobulins of all classes (Tsukada 1993, Vetrie 1993). This indicates the potential for BTK inhibition to suppress production of autoantibodies thought to be important in the development of autoimmune diseases, such as ITP.

While BTK is not expressed in T cells, natural killer cells, or plasma cells and has no traceable direct functions in T cells or plasma cells (Sideras and Smith 1995; Mohamed et al., 2009), the enzyme regulates the activation of other hematopoietic cells, such as B cells, monocytes, basophils, mast cells, macrophages, neutrophils, and platelets. For example, BTK plays a role in the activation of neutrophils, which are key players in the inflammatory response that contributes to wound healing but may also cause tissue damage (Volmering S et al., 2016).

Accordingly, a selective BTK inhibitor has the potential to target multiple pathways involved in inflammation and autoimmunity, including, but not limited to: blocking BCR signaling, B cell activation, and autoantibody production; inhibiting plasma cell differentiation; blocking IgG-mediated FcγR activation, phagocytosis, and inflammatory mediators in monocytes or macrophages; blocking IgE-mediated FcεR activation, migration, and degranulation in mast cells or basophils; and inhibiting activation, adhesion, recruitment, and oxidative burst in neutrophils. Based on these effects, a selective BTK inhibitor may block the initiation and progression of various inflammatory diseases and mitigate tissue damage resulting from these diseases. Although individuals with loss of function mutations in the BTK gene have decreased humoral immunity and are susceptible to pyogenic bacterial and enterovirus infections, requiring treatment with intravenous immunoglobulin, inhibition of BTK in individuals with an intact immune system is not predicted to produce similar susceptibility to infection.

Several orally administered BTK inhibitors (BTKi), including ibrutinib (PCI-32765) and spebrutinib (CC-292), are currently marketed or in clinical development for a range of indications (Lee A et al., 2017). For example, ibrutinib has provided further clinical validation of the BTK target and was recently approved for human use in mantle cell lymphoma, Waldenstrom's macroglobulinemia, and chronic lymphocytic leukemia by the U.S. Food and Drug Administration (FDA). Ibrutinib has also demonstrated activity in other hematological malignancies (Wang 2013; Byrd 2013, Imbruvica Package Insert, 2015). In addition, CC-292 has been reported to be well tolerated in a healthy volunteer population at doses which provide 100% occupancy of the BTK enzyme (Evans 2013). Furthermore, evobrutinib recently demonstrated efficacy for multiple sclerosis in a Phase 2 trial (Montalban X et al., 2019). Other BTKi compounds are in clinical development for various immune-mediated disorders, such as rheumatoid arthritis (NCT03823378, NCT03682705, NCT03233230), and asthma (NCT03944707) (Montalban X et al., 2019; Norman P 2016; Tam C S et al., 2018; Crawford J J et al., 2018; Min T K et al., 2019; Gillooly K M 2017; Nadeem A et al., 2019).

While covalent BTKi, such as ibrutinib and acalabrutinib, improved on the selectivity issues that plagued many first-generation kinase inhibitors, these inhibitors are typically irreversible, causing permanent modification of both on- and off-target kinases and side effects such as thrombocytopenia, anemia, platelet aggregation, and hepatotoxicity (RITUXAN Prescribing Information, 2018; Drug Record Kinase Inhibitors, 2019; Khan Y et al., 2019; Paydas S, 2019; IMBRUVICA, 2013; Rigg R A et al., 2016; Tang C P S et al., 2018). Thus, there is a need for treatment modalities for immune-mediated diseases such as ITP, based on BTKi with reduced side effects.

Compound (I) is a BTK inhibitor of the following structure:

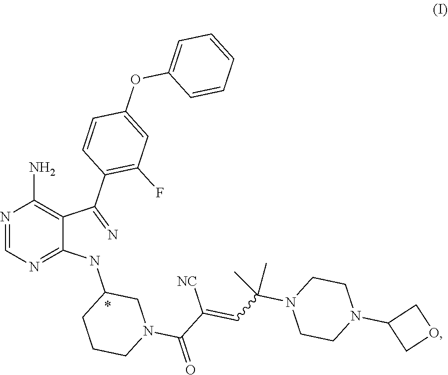 [SMILES: CC(C)(C=C(C#N)C(=O)N1CCCC(Nc2ncnc(N)c2C(=N)c2ccc(Oc3ccccc3)cc2F)C1)N1CCN(C2COC2)CC1]

wherein *C is a stereochemical center. See PCT Publication No. WO 2014/039899, which is incorporated herein by reference, e.g., Example 31.

(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, having the following structure:

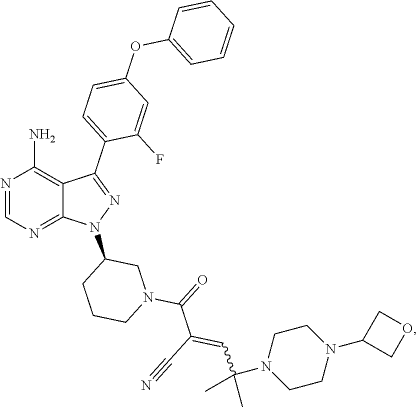 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

is also known as PRN1008 and rilzabrutinib. This compound has been disclosed in several patent publications, such as, e.g., PCT Publication Nos. WO 2014/039899, WO 2015/127310, WO 2016/100914, WO 2016/105531, WO 2018/005849, and WO 2021/150723, the contents of each of which are incorporated by reference herein.

Rilzabrutinib is a novel, highly selective, and potent small molecule inhibitor of non-T cell white blood cell signaling via B-cell receptor, FcγR, and/or FcgR signaling of the BTK pathway. In the context of ITP, rilzabrutinib has the potential to (1) inhibit B cell activation and (2) interrupt antibody-coated cell phagocytosis by FCγR in the spleen and liver (Bradshaw et al. 2021, Langrish et al. 2021, Owens et al. 2022).

Rilzabrutinib functions as a reversible covalent BTK inhibitor and forms both a non-covalent and a covalent bond with its target; in particular, its reversible cysteine binding enables high selectivity and precise BTK inhibition without a permanent modification of proteins and peptides (Langrish et al. 2021, Owens et al. 2022, Smith P F et al. 2017). Taken together, these properties allow for enhanced selectivity and extended inhibition with low systemic exposure. In comparison to first and second generation BTKi, rilzabrutinib has shown minimal cross-reactivity with other molecules and is low risk for off-target effects (Smith P F et al. 2017). Importantly, rilzabrutinib's reversible binding minimizes the likelihood of permanently modified peptides (Serafimova I M 2012). In addition, rilzabrutinib shows improved kinase selectivity relative to the covalent BTK inhibitor ibrutinib. Preclinical studies in a broad kinase enzyme inhibition panel showed that 1 μM rilzabrutinib achieved >90% inhibition of just 6 of 251 kinases sharing a common cysteine in their active site. By contrast, 1 μM ibrutinib inhibited 21 kinases. Rilzabrutinib's IC50 values were 1.3 nM for BTK, 0.8 nM for tyrosine protein kinase TEC, 1.0 nM for bone marrow tyrosine kinase on chromosome X (BMX), 1.2 nM for receptor-like kinase (RLK), 6.3 nM for B cell lymphocyte kinase (BLK), and 11 nM for ERBB4. Further preclinical assays with rilzabrutinib showed that binding to BTK persisted while that for other TEC family members decayed rapidly over time.

Rilzabrutinib has shown encouraging results for the treatment of immune-mediated diseases. In humans, rilzabrutinib is rapidly absorbed following oral administration, with a fast half-life (3-4 h) and variable pharmacokinetics (Smith P F et al., 2017).

In Phase 1 studies of rilzabrutinib with 114 healthy volunteers, target BTK occupancy levels were safely and consistently exceeded, suggesting rilzabrutinib may be highly effective in treating autoimmune diseases. Moreover, preclinical and clinical pharmacokinetic and pharmacodynamic data showed that treatment effects endured even after the compound was cleared from circulation, consistent with an extended target residence time (Hill R et al., 2015) and high target occupancy rate (>90% within four hours and high sustained occupancy over 24 h) (Smith P F et al., 2015).

Rilzabrutinib has also demonstrated a favorable safety profile in clinical studies. In contrast with non-selective, irreversible BTK inhibitors, rilzabrutinib does not alter platelet aggregation in healthy volunteers or patients with ITP and thus does not lead to bleeding problems (Langrish et al. 2021, von Hundelshausen and Seiss 2021). As an additional point of contrast with irreversible BTK inhibitors, rilzabrutinib treatment does not exert clinically relevant effects on cardiac repolarization (electrocardiogram parameters including corrected QT interval) in healthy volunteers (N=51), even when administered at supratherapeutic doses (Lipsky and Lamanna 2020). Indeed, the most commonly reported adverse events in healthy volunteers were gastrointestinal adverse events, including nausea/vomiting and diarrhea. No serious adverse events or deaths were reported, and no participants discontinued treatment due to an adverse event (Smith P F 2017). Moreover, based on preclinical reproductive toxicity studies, rilzabrutinib is not expected to harm fetal development or male fertility.

There is preliminary evidence to support the role of BTK inhibition in patients with autoimmune cytopenias (Rogers 2016, Montillo 2017), where sequential episodes of severe autoimmune hemolytic anemia and ITP ceased after initiation of treatment with ibrutinib, a BTK/EGFR/ITK inhibitor, in patients with chronic lymphatic leukemia. Additionally, and pertinent to the treatment of ITP, rilzabrutinib treatment in vitro profoundly inhibits human B cell activation and blocks antibody (IgG, IgE) mediated activation of immune cells via Fc receptor signaling. In nonclinical studies, rilzabrutinib demonstrates a significant dose dependent reduction of platelet-loss (consumption) in a mouse model of ITP.

In Part A of an international, adaptive, open-label, dose-finding phase 1/2 trial, treatment with rilzabrutinib was generally well tolerated and resulted in a rapid and durable therapeutic effect in ITP patients who were previously treated with multiple therapies. Overall, the 400 mg twice-daily dose of rilzabrutinib was shown to be safe and effective.

Part B of the open-label, phase 1/2 study continued to evaluate the safety and efficacy of 400 mg BID rilzabrutinib in patients with relapsed ITP. Adult patients (aged 18-80 years) with ≥2 baseline platelet counts <30×10⁹/μL no less than 7 days apart in the 15 days before the first dose. Eligible patients were required to have a past response (achievement of platelet count ≥50×10⁹/μL) to IVIG/anti-D or CS that was not sustained, and to have failed ≥1 other non-IVIG, non-CS ITP therapy. Stable doses of concomitant CS/TPO-RA were allowed with rilzabrutinib. The primary endpoints were safety and durable platelet response (defined as platelet counts ≥50×10⁹/μL on ≥8 of the last 12 weeks of rilzabrutinib without rescue medication). Consistent with the results from Part A, rilzabrutinib demonstrated rapid, stable, and durable platelet responses in patients with relapsed ITP, with a favorable safety profile in part B. In particular, patients treated with rilzabrutinib maintained median platelet counts ≥50×10⁹/μL throughout the main treatment period and long-term extension (LTE) (FIG. 1).

In Parts A and B, treatment with rilzabrutinib also led to improvements in patient Platelet Variability Index (PVI), a recently developed metric for capturing the degree of platelet count fluctuations and thrombocytopenia severity during a specified time period (Li et al. 2021). PVI scores, which range from 0 (indicating low disease activity) to 6 (indicating high disease activity) were determined in aggregate from parts A and B using platelet count values obtained during the following time periods: baseline, the main treatment period (weeks 5, 9, 13, 17, 21, and 25), and the LTE (weeks 29, 33, 37, 41, 45, and 49).

Overall, 71 patients with ITP received rilzabrutinib 400 mg BID, either alone (n=24) or with concomitant ITP therapy (n=47). For all patients at baseline, the median PVI score was 2 (interquartile range (IQR): 2, 3), while the median platelet count was 14×10⁹/μL (IQR: 6×10⁹/μL, 20×10⁹/μL). Among 29 (41%) responding patients (defined as patients who achieved ≥2 consecutive platelet counts ≥50×10⁹/μL and increased ≥20×10⁹/μL from baseline without rescue medication (per the part A primary endpoint)), the median PVI during the main treatment period was 2 (IQR 1, 3), whereas among 42 (59%) non-responding patients, the median PVI during the main treatment period increased to 3 (IQR: 2, 3; FIG. 2). Additionally, for responding patients during the LTE period, median PVI scores decreased to 1 (IQR: 0, 2). When evaluated based on treatment with rilzabrutinib alone or in combination with concomitant ITP therapy in the 29 responding patients, median PVI scores decreased from baseline to the LTE period whether or not patients received concomitant ITP therapy (FIG. 3).

Platelet counts increased in both responders and non-responders. Among responding patients, baseline median platelet counts of 18×10⁹/μL rising to 76×10⁹/μL during the main treatment period, and to 89×10⁹/μL during the LTE. Meanwhile, in non-responding patients, baseline median platelet counts of 11×10⁹/μL also increased slightly to 17×10⁹/μL during the main treatment period.

Moreover, many of the 71 patients who initiated rilzabrutinib 400 mg BID were early responders. Of the 29 (41%) responding patients, 21/29 (72%) were early responders, defined as achieving platelet counts ≥30×10⁹/μL at week 2. Significant predictors of both response and early response to rilzabrutinib based on univariate logistic regression models were higher baseline platelet counts and no prior use of TPO-RA (p≤0.05). Conversely, the use of concomitant ITP medication with TPO-RAs was not associated with predicting either response or early response to rilzabrutinib.

Assessment of continuous variables by response outcome showed that responders (vs. non-responders) had a numerically shorter duration of ITP (8.9 vs. 14.3 years; p=0.07), significantly fewer prior therapies (7 vs. 13; p=0.03), and significantly higher baseline platelet counts (17×10⁹/μL vs 12×10⁹/μL; p=0.01; FIGS. 4A-B). Similarly, early responders (vs. non-early responders) had significantly shorter duration of ITP (7.4 vs. 14.1 years; p=0.01), fewer prior ITP therapies (7 vs. 12; p=0.03), and higher baseline platelet counts (19×10⁹/μL vs 12×10⁹/μL; p=0.002). When the continuous variables were assessed on the basis of prior TPO-RA use, patients with no prior TPO-RA use (vs. prior TPO-RA use) had a significantly shorter duration of ITP (7.4 vs. 13.7 years; p=0.04), fewer prior ITP therapies (5 vs. 12; p=0.0008), and higher baseline platelet counts (17×10⁹/μL vs 13×10⁹/μL; p=0.03).

Across categorical variables, significant associations were observed with being a responder (vs. non-responder) and no prior use of TPO-RAs (p=0.01). Both no prior TPO-RA use (p=0.0008) and no prior rituximab use (p=0.04) were significantly associated with improved early response.

Change from baseline in the ITP Bleeding Scale (IBLS) score (0 none to 2 marked bleeding) was a secondary endpoint to assess bleeding across 9 anatomical sites (8 anatomical sites for male/postmenopausal women). An exploratory health-related quality of life (HRQOL) endpoint evaluated the EuroQol-5 Dimensions 5-Level (EQ-5D-5L)+Visual Analog Scale (EQ-VAS) and ITP Patient Assessment Questionnaire™ (ITP-PAQ) scores (0 worst to 100 best QOL). Overall, patients treated with rilzabrutinib exhibited a durable platelet responses, high compliance, and improvements on HRQOL measures in difficult to treat patients with relapsed ITP. There was no evidence of increased bleeding with rilzabrutinib, with scores improving at the skin site. Clinically meaningful improvements in HRQOL were observed in multiple individual and overall HRQOL health domains following rilzabrutinib (FIG. 5).

The 400 mg BID dose is being assessed in an ongoing LUNA 3 multicenter, double-blind, placebo-controlled, phase 3 study. The primary endpoint is durable platelet response, defined as (1) the achievement of platelet counts of ≥50×10⁹/μL for at least two-thirds of ≥8 available, weekly scheduled platelet measurements during the last 12 weeks of the 24-week blinded treatment period in the absence of rescue therapy (e.g., in 8 of 12 or 6 of 9 counts) and provided that at least 2 available weekly scheduled platelet measurements are at or above 50,000/μL during the last 6 weeks of the 24-week blinded treatment period; or (2) the achievement of platelet counts of ≥50×10⁹/μL for ≥8 out of the 12 schedule observations in the last 12 weeks of the 24-week double-blinded treatment period in the absence of rescue therapy (EU and UK). The LUNA 3 trial will further investigate the magnitude and durability of rilzabrutinib's safety and efficacy in adult and pediatric patients.

Disclosed herein are methods for treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has or has been identified as having an initial platelet count of <30,000/μL with no single platelet count >35,000/μL in the 2 weeks prior to the treatment period, and further wherein the human patient has or has been identified as having at least one characteristic chosen from: a prior response to one or more of IVIg, anti-D, or CSs that was not sustained; a documented intolerance or insufficient response to any appropriate courses of standard-of-care ITP therapies; and a contraindication for any appropriate courses standard-of-care ITP therapies. In some embodiments, the human patient has a prior response to one or more of IVIg, anti-D, or CSs that was not sustained. In some embodiments, the human patient has a documented intolerance or insufficient response to any appropriate courses of standard-of-care ITP therapies. In some embodiments, the human patient has a contraindication for any appropriate courses of standard-of-care ITP therapies. In some embodiments, the human patient has a prior response to one or more of IVIg, anti-D, or CSs that was not sustained and a documented intolerance or insufficient response to any appropriate courses of standard-of-care ITP therapies. In some embodiments, the patient has a prior response to one or more of IVIg, anti-D, or CSs that was not sustained and a contraindication for any appropriate courses of standard-of-care ITP therapies. In some embodiments, the human patient has a documented intolerance or insufficient response to any appropriate courses of standard-of-care ITP therapies and a contraindication for any appropriate courses of standard-of-care ITP therapies. In some embodiments, the human patient has a prior response to one or more of IVIg, anti-D, or CSs that was not sustained; a documented intolerance or insufficient response to any appropriate courses of standard-of-care ITP therapies; and a contraindication for any appropriate courses of standard-of-care ITP therapies.

Disclosed herein are methods for treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient has or has been identified as having had an insufficient response or intolerance to a previous treatment or to previous treatments.

Disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for treating immune thrombocytopenia (ITP) in a human patient with persistent or chronic ITP in need thereof.

Disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for use as a medicament for treating immune thrombocytopenia (ITP) in a human patient with persistent or chronic ITP in need thereof.

Disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for use for treating immune thrombocytopenia (ITP) in a human patient with persistent or chronic ITP in need thereof.

Disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, as a medicament for treating immune thrombocytopenia (ITP) in a human patient with persistent or chronic ITP in need thereof.

### BRIEF DESCRIPTION OF THE DRAWINGS

FIG. 1 shows median platelet counts over the course of the main treatment period and LTE.

FIG. 2 shows the median (IQR) PVI and platelet counts by response to rilzabrutinib at baseline, during the main treatment period, and during the LTE. *Non-responders did not qualify for LTE entry (LTE criteria included platelet counts ≥50×10⁹/μL for ≥50% of last 8 weeks of treatment).

FIG. 3 shows the median (IQR) PVI score in responding patients who received rilzabrutinib alone or with concomitant ITP therapy at baseline, during the main treatment, and during the LTE. Note: there were two fewer patients in all groups except rilzabrutinib with TPO-RA/CS during the LTE period.

FIGS. 4A-B show predictors of response to rilzabrutinib. (A) shows a univariate logistic regression model of pooled predictors of response to rilzabrutinib. *Due to low patient numbers, the model did not fit for ITP diagnosis group and no prior CS use. (B) shows a summary of continuous variables by response to rilzabrutinib or prior TPO-RA use. Horizontal bars indicate the mean+1 SD. *Signifies an improved response with p<0.05 by two sample t-test assuming unequal variances.

FIG. 5 shows the median change from baseline HRQOL to Week 25 HRQOL during Part B of the phase 1/2 study. MID references provided in Mathias S D, et al. CMRO. 2009; 25(2):375-383.

FIG. 6 shows the study schema as a flow chart. ‡Week 25 is the last visit of the blinded treatment period and also serves as the start of the open-label period. †Following long-term extension completion, patients will undergo last day of study drug and end of study assessments.

FIG. 7 shows the decision flow chart for assessing response at Week 13. After enrollment, patients will start a blinded treatment period for up to 24 weeks (rilzabrutinib or placebo treatment) followed by an open-label period of 28 weeks (all patients receive rilzabrutinib), and then a 4-week safety follow-up period or LTE. Patients who do not complete the initial 12 weeks of treatment are not eligible to proceed to the open-label period. At the end of 12 weeks of treatment, participants will be assessed for platelet response (≥50×10⁹/μL or between ≥30×10⁹/μL and <50×10⁹/μL and at least doubled from baseline at any time) and the presence or absence of rescue medication in the 4 weeks before elevated platelet counts meeting the platelet response criteria. Responders with no rescue medication use after week 8 will continue to be evaluated for eligibility to proceed to the blinded treatment period for a total of 24 weeks before entering the open-label period. Non-responders or those who did receive rescue medication after week 8 may discontinue the study or enter the 28-week open-label period at the end of week 12 and receive rilzabrutinib 400 mg BID.

FIG. 8 shows the percentage of patients in the placebo and rilzabrutinib arms who achieved a durable platelet response over the course of the 24-week double-blinded period.

FIG. 9 shows the mean number of weeks with a platelet count ≥50×10⁹/μL or between ≥30×10⁹/μL and <50×10⁹/μL and at least doubled from baseline during the 24-week blinded treatment period in the absence of rescue medication.

FIG. 10 shows the time to first platelet count ≥50×10⁹/μL or time to first platelet count between ≥30×10⁹/μL and ≤50×10⁹/μL and at least doubled from baseline.

FIG. 11 shows the proportion of patients requiring rescue therapy during the 24-week blinded treatment period.

FIG. 12 shows the change from baseline on ITP-PAQ™ physical fatigue score in adult patients (≥18 years of age) at Week 13 of treatment.

FIG. 13 shows the change in physical fatigue over time up to Week 25.

FIG. 14 shows the change in bleeding as assessed by IBLS at Week 25.

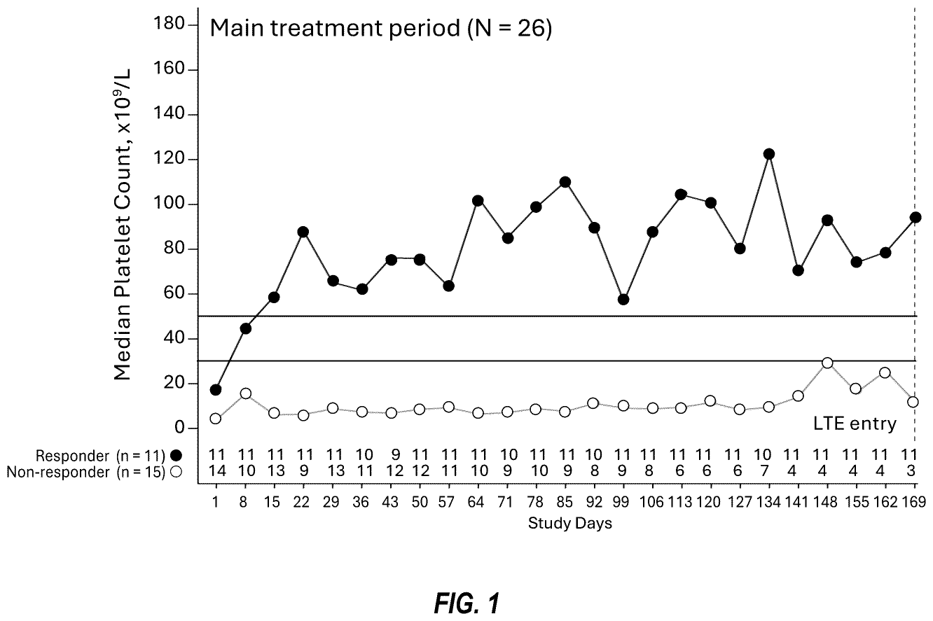

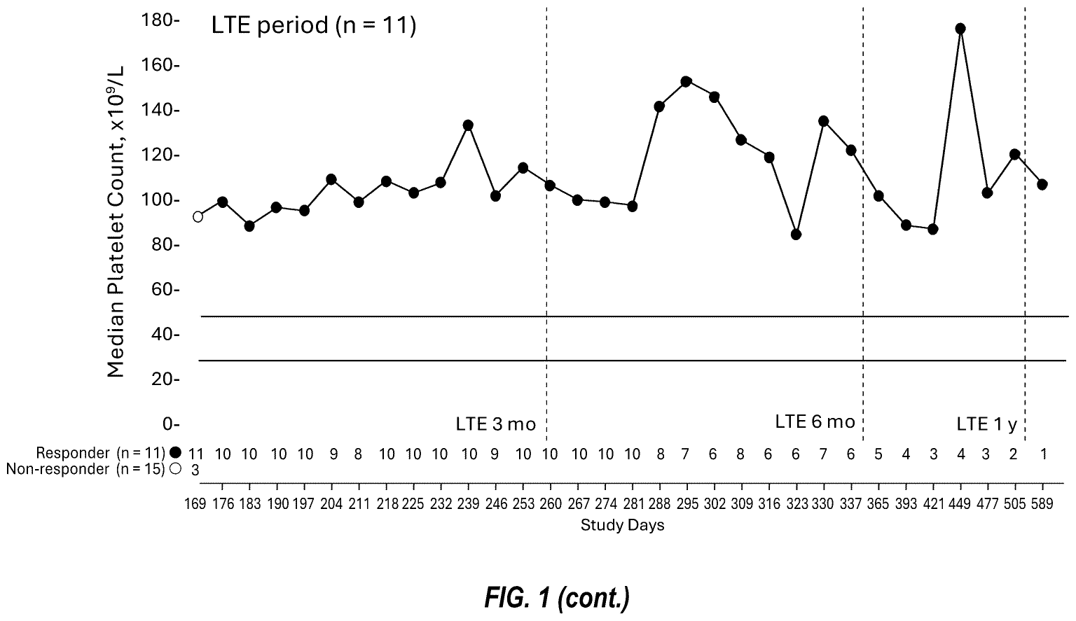

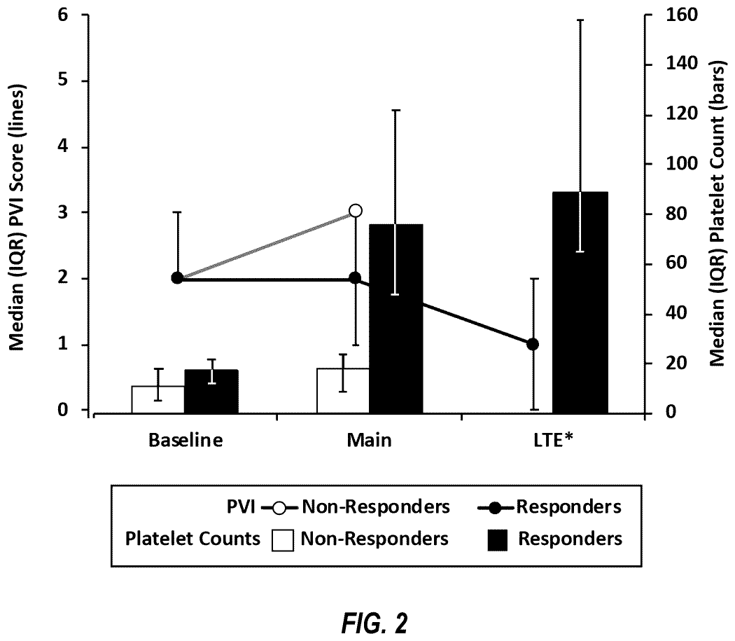

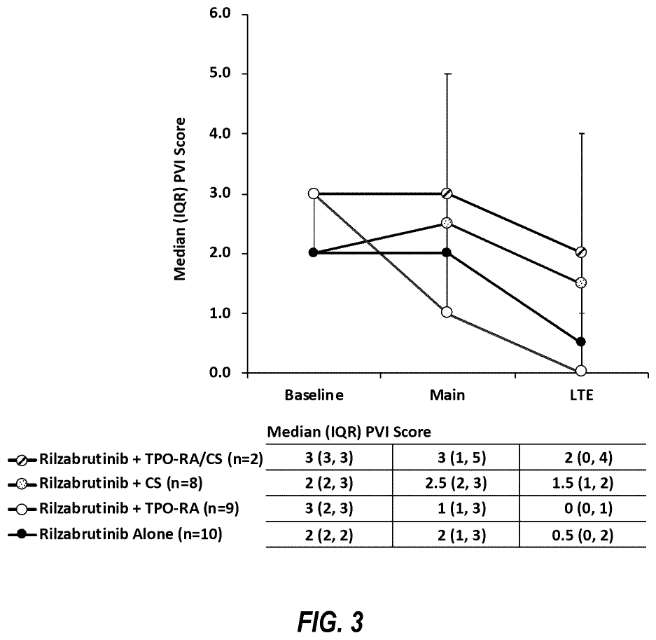

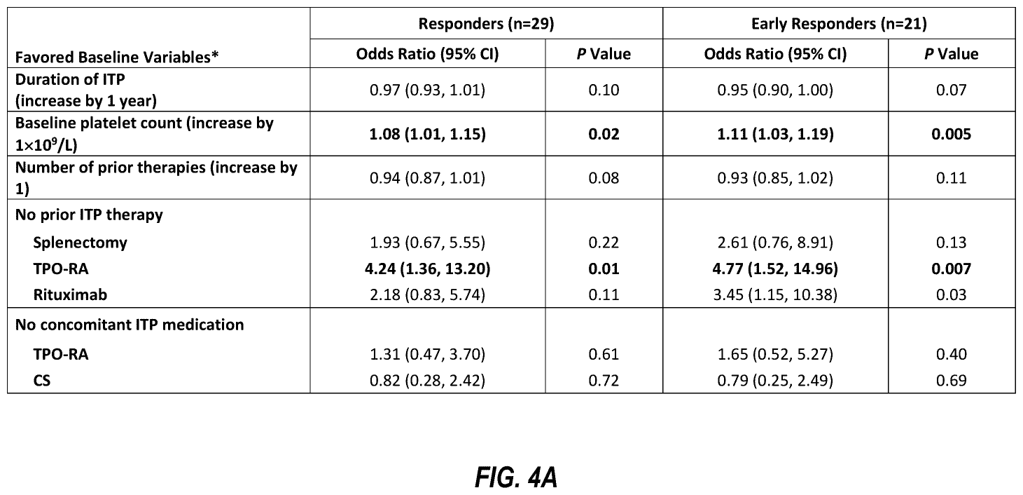

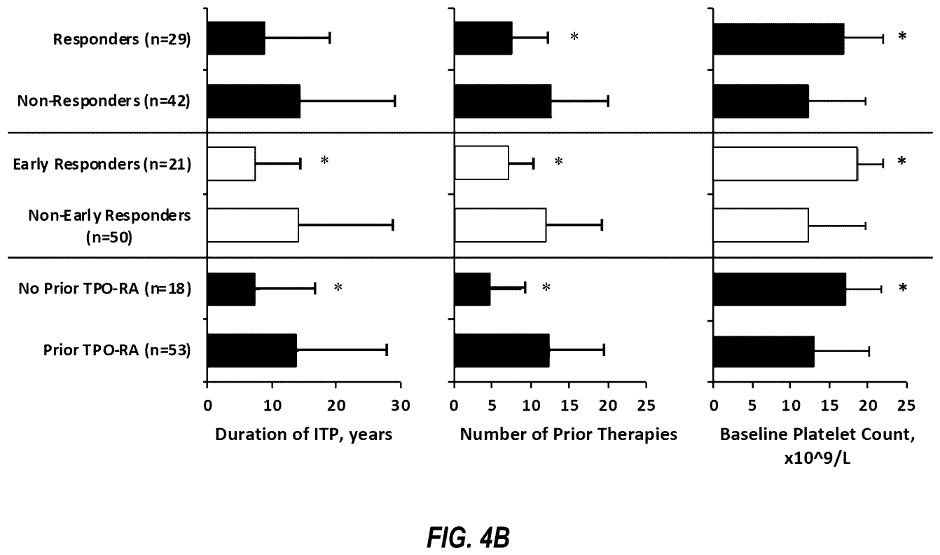

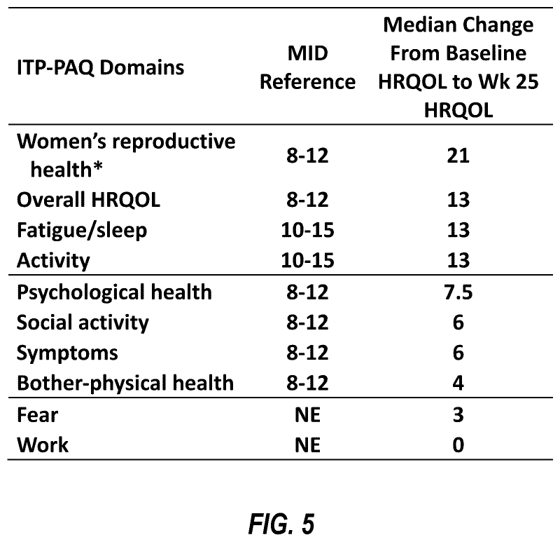

### DEFINITIONS

Unless otherwise stated, the following terms used in the specification and claims are defined for the purposes of this Application and have the following meanings. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

As used herein, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. With regard to specific values, it should be understood that specific values described herein for subject populations (e.g., the subject of the described clinical trial) represent median, mean, or statistical numbers, unless otherwise provided. Accordingly, aspects of the present disclosure requiring a particular value in a subject are supported herein by population data in which the relevant value is assessed to be a meaningful delimitation on the subject population.

As used herein, the term “active pharmaceutical ingredient” or “therapeutic agent” (“API”) refers to a biologically active compound.

As used herein, “adolescent” and “adolescents” encompass all patients aged 12 to <18 years.

As used herein, the term “approved treatment” refers to a medication that has received regulatory authorization, in any country, for its intended use.

As used herein, the terms “administer,” “administering,” or “administration” herein refer to providing, giving, dosing, and/or prescribing by either a health practitioner or an authorized agent and/or putting into, taking, or consuming by the patient or person himself or herself. For example, “administration” of an API to a patient refers to any route (e.g., oral delivery) of introducing or delivering the API to the patient. Administration includes self-administration and administration by another.

As used herein, the terms “baseline platelet count” or “baseline” refer to an average platelet count obtained by determining the mean of 2 platelet counts taken on 2 occasions no less than 7 days apart in the 15 days prior to beginning treatment and a third count taken on the first day of the study. If any of these three counts is missing, the “baseline platelet count” or “baseline” is the average of the other counts.

As used herein, “BID” and “bid” are used interchangeably to refer to twice a day.

As used herein, “child” and “children” encompass all patients aged <12 years. In some embodiments, a child may be aged 0 to <12 years. In some embodiments, a child may be aged 10 to <12 years.

As used herein, “immune thrombocytopenia” (ITP) encompasses or at least also refers to other terms commonly used such as idiopathic thrombocytopenia and idiopathic thrombocytopenic purpura. There are three main types of ITP: Acute (newly diagnosed), persistent, and chronic (long term). Acute ITP lasts less than three months, persistent ITP lasts 3-12 months, and chronic ITP lasts for at least one year.

As used herein, the terms “initial platelet count” or “initial count” refer to an average platelet count obtained by determining the mean of at least 2 platelet counts at least 5 days apart prior to treatment.

As used herein, “ITP Kids' ITP Tools” and “ITP-KIT” are used interchangeably to refer to a battery of three disease-specific instruments: a child self-report form designed to be completed by pediatric patients ≥7 years, a parent proxy report form for pediatric patients <7, and a parent impact form. Respondents record their disease experience based on a 1-week recall. The instrument yields a total score which is the summation of the items converted to a 0 to 100 score with higher scores indicating better disease-specific Quality of Life (QOL).

As used herein, “ITP Patient Assessment Questionnaire™” and “ITP-PAQ™” are used interchangeably to refer to a disease-specific instrument that was designed to measure the Quality of Life (QOL) of adult patients with immune thrombocytopenia. The items employ a 4-week recall with responses recorded on 4-, 5- or 7-point Likert scales. All item scores are transformed to a 0 to 100 continuum where higher scores represent better QoL and are weighted equally to derive the scale scores.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

As used herein, an amount expressed in terms of “mg of [X]” refers to the total amount in milligrams of [X], i.e., the free base. In some embodiments, rilzabrutinib may be administered as a pharmaceutically acceptable salt of rilzabrutinib, in which case an amount expressed in terms of “mg of rilzabrutinib” refers to the total amount in milligrams of rilzabrutinib, i.e., the free base, plus the equivalent amount of one or more pharmaceutically acceptable salts of rilzabrutinib based on the weight of free base therein. For example, “400 mg of at least one compound chosen from rilzabrutinib and pharmaceutically acceptable salts thereof” includes 400 mg of rilzabrutinib and a concentration of one or more pharmaceutically acceptable salts of rilzabrutinib equivalent to 400 mg of rilzabrutinib.

As used herein, a “pediatric patient” is a patient aged <18 years. In some embodiments, a pediatric patient may be aged 0 to <18 years. In some embodiments, a pediatric patient may be aged 10 to <18 years. In some embodiments, a pediatric patient may be aged 12 to <18 years.

As used herein, a “pharmaceutically acceptable carrier or excipient” means a carrier or an excipient that is useful in preparing a pharmaceutical composition that is generally safe, and neither biologically nor otherwise undesirable, such as, e.g., a carrier or an excipient that is acceptable for mammalian pharmaceutical use.

As used herein, the term “pharmaceutically acceptable salt” refers to a salt form, e.g., an acid addition salt, of an active pharmaceutical agent that is pharmaceutically acceptable and that possesses the desired pharmacological activity of the API of which the salt is made. Pharmaceutically acceptable salts are well known in the art and include those derived from suitable inorganic and organic acids. Such salts include, but are not limited to, salts formed with inorganic acids such as hydrochloric acid, hydrobromic acid, sulfuric acid, phosphoric acid, and the like; or formed with organic acids such as formic acid, acetic acid, propionic acid, hexanoic acid, lactic acid, malonic acid, succinic acid, malic acid, maleic acid, fumaric acid, tartaric acid, citric acid, benzoic acid, mandelic acid, methanesulfonic acid, ethanesulfonic acid, 1,2-ethanedisulfonic acid, benzenesulfonic acid, 4-toluenesulfonic acid, and the like. S. M. Berge et al. describes pharmaceutically acceptable salts in detail in J. Pharmaceutical Sciences, 1977, 66, 1-19.

As used herein, the terms “PRN1008,” “rilzabrutinib,” “(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile;” “the compound of Formula (I);” and “2-[(3R)-3-[4-amino-3-(2-fluoro-4-phenoxy-phenyl)pyrazolo[3,4-d]-pyrimidin-1-yl]piperidine-1-carbonyl]-4-methyl-4-[4-(oxetan-3-yl)piperazin-1-yl]pent-2-enenitrile” are used interchangeably to refer to a compound having the structure:

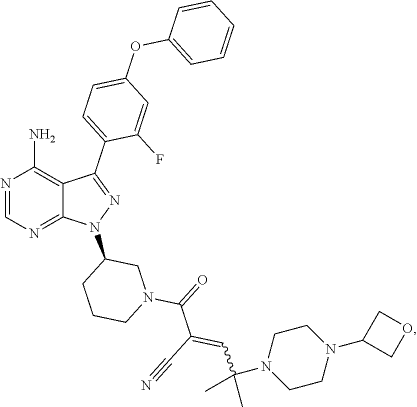 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

which is also referred to as 2-[(3R)-2-[4-amino-3-(2-fluoro-4-phenoxy-phenyl)pyrazolo[3,4-d]pyrimidin-1-yl]piperdine-1-carbonyl]-4-methyl-4[4-(oxetan-3-yl)piperazin-1-yl]-(E and Z)-pent-2-enenitrile; (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; 1-piperidinepropanenitrile, 3-[4-amino-3-(2-fluoro-4-phenoxyphenyl)-1H-pyrazolo[3,4-d]pyrimidin-1-yl]-α-[2-methyl-2-[4-(3-oxetanyl)-1-piperazinyl]propylidene]-3-oxo-, (3R)-; (EZ)-2-[(3R)-3-[4-amino-3-(2-fluoro-4-phenoxyphenyl)pyrazolo[3,4-d]pyrimidin-1-yl]piperidine-1-carbonyl]-4-methyl-4[4-(oxetan-3-yl)piperazin-1-yl]pent-2-enenitrile; and also by the International Nonproprietary Names for Pharmaceutical Substances (INN) as published by the World Health Organization (https://cdn.who.int/media/docs/default-source/international-nonproprietary-names-(inn)/pl121.pdf?sfvrsn=69617906_15&download=true) having the following structure:

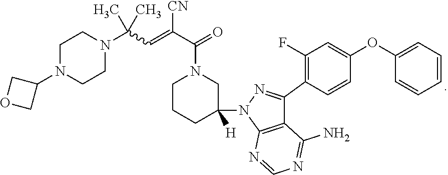 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

The compound of Formula (I) includes E and Z isomers, as indicated by the wavy bond in the structure shown above. The compound of Formula (I) may be present as a salt form.

A dose of the (E) isomer of rilzabrutinib may contain the corresponding (Z) isomer as an impurity in less than about 1% by weight; a dose of the (Z) isomer of rilzabrutinib may contain the corresponding (E) isomer as an impurity in less than about 1% by weight. When rilzabrutinib is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 1% by weight. In some embodiments, the molar ratio of (E) to (Z) isomer is 9:1. Rilzabrutinib or a pharmaceutically acceptable salt thereof may also be referred to herein as a “drug,” “active agent,” “a therapeutically active agent,” or “API.”

As used herein, “QD” and “qd” are used interchangeably to refer to once a day.

As used herein, the term “therapeutically effective amount” refers to that of a compound that produces the desired effect for which it is administered (e.g., improvement in ITP or a symptom of ITP, or lessening the severity of ITP or a symptom of ITP). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

As used herein, the term “response” refers to a change in platelet levels following the administration of any appropriate standard-of-care ITP therapy.

Some embodiments of the present disclosure relate to a method for treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has or has been identified as having an initial platelet count of <30,000/μL with no single platelet count >35,000/μL in the 2 weeks prior to the treatment period, and further wherein the human patient has or has been identified as having at least one characteristic chosen from: a prior response to one or more of IVIg, anti-D, or CSs that was not sustained; a documented intolerance or insufficient response to any appropriate courses of standard-of-care ITP therapies; and a contraindication for any appropriate courses standard-of-care ITP therapies. In some embodiments, the human patient has a prior response to one or more of IVIg, anti-D, or CSs that was not sustained. In some embodiments, the human patient has a documented intolerance or insufficient response to any appropriate courses of standard-of-care ITP therapies. In some embodiments, the human patient has a contraindication for any appropriate courses of standard-of-care ITP therapies. In some embodiments, the human patient has a prior response to one or more of IVIg, anti-D, or CSs that was not sustained and a documented intolerance or insufficient response to any appropriate courses of standard-of-care ITP therapies. In some embodiments, the patient has a prior response to one or more of IVIg, anti-D, or CSs that was not sustained and a contraindication for any appropriate courses of standard-of-care ITP therapies. In some embodiments, the human patient has a documented intolerance or insufficient response to any appropriate courses of standard-of-care ITP therapies and a contraindication for any appropriate courses of standard-of-care ITP therapies. In some embodiments, the human patient has a prior response to one or more of IVIg, anti-D, or CSs that was not sustained; a documented intolerance or insufficient response to any appropriate courses of standard-of-care ITP therapies; and a contraindication for any appropriate courses of standard-of-care ITP therapies.

In some embodiments, the human patient has or has been identified as having an initial platelet count of <15,000/μL. In some embodiments, the human patient has or has been identified as having an initial platelet count of <10,000/μL. In some embodiments, the human patient has or has been identified as having an initial platelet count of <5,000/μL. In some embodiments, the human patient has or has been identified as having an initial platelet count of <4,000/μL. In some embodiments, the human patient has or has been identified as having an initial platelet count of <3,000/μL. In some embodiments, the human patient has or has been identified as having an initial platelet count of <2,000/μL. In some embodiments, the human patient has or has been identified as having an initial platelet count of <1,000/μL.

In some embodiments, the human patient has or has been identified as having an initial platelet count of ≥15,000/μL. In some embodiments, the human patient has or has been identified as having an initial platelet count of ≥20,000/μL. In some embodiments, the human patient has or has been identified as having an initial platelet count of ≥25,000/μL. In some embodiments, the human patient has or has been identified as having an initial platelet count of ≥30,000/μL.

In some embodiments, the human patient achieves a platelet response.

In some embodiments, the human patient achieves a durable platelet response.

In some embodiments, the human patient achieves a complete platelet response.

In some embodiments, the human patient achieves a stable platelet response.

In some embodiments, the human patient has at least one platelet count of ≥30,000/μL during the treatment period. In some embodiments, the human patient has at least one platelet count of ≥35,000/μL during the treatment period. In some embodiments, the human patient has at least one platelet count of ≥40,000/μL during the treatment period. In some embodiments, the human patient has at least one platelet count of ≥45,000/μL during the treatment period.

In some embodiments, the human patient has at least one platelet count of ≥30,000/μL and at least a doubling of the baseline platelet count during the treatment period. In some embodiments, the human patient has at least one platelet count of ≥35,000/μL and at least a doubling of the baseline platelet count during the treatment period. In some embodiments, the human patient has at least one platelet count of ≥40,000/μL and at least a doubling of the baseline platelet count during the treatment period. In some embodiments, the human patient has at least one platelet count of ≥45,000/μL and at least a doubling of the baseline platelet count during the treatment period.

In some embodiments, wherein the human patient has at least one platelet count of ≥30,000/μL and at least a doubling of the baseline platelet count during the treatment period, the patient achieves the at least one platelet count within 1 week of initiating treatment. In some embodiments, the patient achieves the at least one platelet count within 2 weeks of initiating treatment. In some embodiments, the patient achieves the at least one platelet count within 3 weeks of initiating treatment. In some embodiments, the patient achieves the at least one platelet count within 4 weeks of initiating treatment. In some embodiments, the patient achieves the at least one platelet count within 5 weeks of initiating treatment. In some embodiments, the patient achieves the at least one platelet count within 6 weeks of initiating treatment. In some embodiments, the patient achieves the at least one platelet count within 7 weeks of initiating treatment. In some embodiments, the patient achieves the at least one platelet count within 8 weeks of initiating treatment. In some embodiments, the patient achieves the at least one platelet count within 9 weeks of initiating treatment. In some embodiments, the patient achieves the at least one platelet count within 10 weeks of initiating treatment. In some embodiments, the patient achieves the at least one platelet count within 11 weeks of initiating treatment. In some embodiments, the patient achieves the at least one platelet count within 12 weeks of initiating treatment. In some embodiments, the patient achieves the at least one platelet count within 13 weeks of initiating treatment. In some embodiments, the patient achieves the at least one platelet count within 14 weeks of initiating treatment. In some embodiments, the patient achieves the at least one platelet count within 15 weeks of initiating treatment. In some embodiments, the patient achieves the at least one platelet count within 16 weeks of initiating treatment. In some embodiments, the patient achieves the at least one platelet count within 17 weeks of initiating treatment. In some embodiments, the patient achieves the at least one platelet count within 18 weeks of initiating treatment. In some embodiments, the patient achieves the at least one platelet count within 19 weeks of initiating treatment. In some embodiments, the patient achieves the at least one platelet count within 20 weeks of initiating treatment. In some embodiments, the patient achieves the at least one platelet count within 21 weeks of initiating treatment. In some embodiments, the patient achieves the at least one platelet count within 22 weeks of initiating treatment. In some embodiments, the patient achieves the at least one platelet count within 23 weeks of initiating treatment. In some embodiments, the patient achieves the at least one platelet count within 24 weeks of initiating treatment.

In some embodiments, the human patient has at least one platelet count of ≥30,000/μL and at least a doubling of the baseline platelet count in the absence of rescue medication during the treatment period. In some embodiments, the human patient has at least one platelet count of ≥35,000/μL and at least a doubling of the baseline platelet count in the absence of rescue medication during the treatment period. In some embodiments, the human patient has at least one platelet count of ≥40,000/μL and at least a doubling of the baseline platelet count in the absence of rescue medication during the treatment period. In some embodiments, the human patient has at least one platelet count of ≥45,000/μL and at least a doubling of the baseline platelet count in the absence of rescue medication during the treatment period.

In some embodiments, the human patient has at least one platelet count of ≥30,000/μL and at least a doubling of the baseline platelet count during a 24-week treatment period. In some embodiments, the human patient has at least one platelet count of ≥35,000/μL and at least a doubling of the baseline platelet count during a 24-week treatment period. In some embodiments, the human patient has at least one platelet count of ≥40,000/μL and at least a doubling of the baseline platelet count during a 24-week treatment period. In some embodiments, the human patient has at least one platelet count of ≥45,000/μL and at least a doubling of the baseline platelet count during a 24-week treatment period.

In some embodiments, the human patient has at least one platelet count of ≥30,000/μL and at least a doubling of the baseline platelet after a 6-month treatment period. In some embodiments, the human patient has at least one platelet count of ≥35,000/μL and at least a doubling of the baseline platelet after a 6-month treatment period. In some embodiments, the human patient has at least one platelet count of ≥40,000/μL and at least a doubling of the baseline platelet after a 6-month treatment period. In some embodiments, the human patient has at least one platelet count of ≥45,000/μL and at least a doubling of the baseline platelet after a 6-month treatment period.

In some embodiments, the human patient has at least one platelet count ≥50,000/μL during the treatment period. In some embodiments, the human patient has at least one platelet count ≥55,000/μL during the treatment period. In some embodiments, the human patient has at least one platelet count ≥60,000/μL during the treatment period. In some embodiments, the human patient has at least one platelet count ≥65,000/μL during the treatment period. In some embodiments, the human patient has at least one platelet count ≥70,000/μL during the treatment period. In some embodiments, the human patient has at least one platelet count ≥75,000/μL during the treatment period. In some embodiments, the human patient has at least one platelet count ≥80,000/μL during the treatment period. In some embodiments, the human patient has at least one platelet count ≥85,000/μL during the treatment period. In some embodiments, the human patient has at least one platelet count ≥90,000/μL during the treatment period. In some embodiments, the human patient has at least one platelet count ≥95,000/μL during the treatment period.

In some embodiments, the human patient has at least one platelet count ≥50,000/μL in the absence of rescue medication during the treatment period. In some embodiments, the human patient has at least one platelet count ≥55,000/μL in the absence of rescue medication during the treatment period. In some embodiments, the human patient has at least one platelet count ≥60,000/μL in the absence of rescue medication during the treatment period. In some embodiments, the human patient has at least one platelet count ≥65,000/μL in the absence of rescue medication during the treatment period. In some embodiments, the human patient has at least one platelet count ≥70,000/μL in the absence of rescue medication during the treatment period. In some embodiments, the human patient has at least one platelet count ≥75,000/μL in the absence of rescue medication during the treatment period. In some embodiments, the human patient has at least one platelet count ≥80,000/μL in the absence of rescue medication during the treatment period. In some embodiments, the human patient has at least one platelet count ≥85,000/μL in the absence of rescue medication during the treatment period. In some embodiments, the human patient has at least one platelet count ≥90,000/μL in the absence of rescue medication during the treatment period. In some embodiments, the human patient has at least one platelet count ≥95,000/μL in the absence of rescue medication during the treatment period.

In some embodiments, the human patient has at least 2 consecutive platelet counts of ≥50,000/μL, wherein the platelet counts are ≥5 days apart, in the absence of rescue medication during the treatment period. In some embodiments, the human patient has at least 2 consecutive platelet counts of ≥55,000/μL, wherein the platelet counts are ≥5 days apart, in the absence of rescue medication during the treatment period. In some embodiments, the human patient has at least 2 consecutive platelet counts of ≥60,000/μL, wherein the platelet counts are ≥5 days apart, in the absence of rescue medication during the treatment period. In some embodiments, the human patient has at least 2 consecutive platelet counts of ≥65,000/μL, wherein the platelet counts are ≥5 days apart, in the absence of rescue medication during the treatment period. In some embodiments, the human patient has at least 2 consecutive platelet counts of ≥70,000/μL, wherein the platelet counts are ≥5 days apart, in the absence of rescue medication during the treatment period. In some embodiments, the human patient has at least 2 consecutive platelet counts of ≥75,000/μL, wherein the platelet counts are 5 days apart, in the absence of rescue medication during the treatment period. In some embodiments, the human patient has at least 2 consecutive platelet counts of ≥80,000/μL, wherein the platelet counts are ≥5 days apart, in the absence of rescue medication during the treatment period. In some embodiments, the human patient has at least 2 consecutive platelet counts of ≥85,000/μL, wherein the platelet counts are ≥5 days apart, in the absence of rescue medication during the treatment period. In some embodiments, the human patient has at least 2 consecutive platelet counts of ≥90,000/μL, wherein the platelet counts are ≥5 days apart, in the absence of rescue medication during the treatment period. In some embodiments, the human patient has at least 2 consecutive platelet counts of ≥95,000/μL, wherein the platelet counts are ≥5 days apart, in the absence of rescue medication during the treatment period.

In some embodiments, the human patient has platelet counts of ≥50,000/μL for at least two-thirds of at least 8 available weekly platelet counts during the last 12 weeks of a 24-week treatment period, further wherein: at least 2 available weekly platelet counts are ≥50,000/μL during the last 6 weeks of a 24-week treatment period; and the human patient does not require rescue medication. In some embodiments, the human patient has platelet counts of ≥50,000/μL for at least 8 available weekly platelet counts during the last 12 weeks of a 24-week treatment period, further wherein the patient does not require rescue medication. In some embodiments, the human patient has platelet counts of ≥50,000/μL for 12 platelet counts during the last 12 weeks of the 24-week treatment period. In some embodiments, at least 2 available weekly platelet counts are ≥55,000/μL during the last 6 weeks of a 24-week treatment period. In some embodiments, at least 2 available weekly platelet counts are ≥60,000/μL during the last 6 weeks of a 24-week treatment period. In some embodiments, at least 2 available weekly platelet counts are ≥65,000/μL during the last 6 weeks of a 24-week treatment period. In some embodiments, at least 2 available weekly platelet counts are ≥70,000/μL during the last 6 weeks of a 24-week treatment period. In some embodiments, at least 2 available weekly platelet counts are ≥75,000/μL during the last 6 weeks of a 24-week treatment period. In some embodiments, at least 2 available weekly platelet counts are ≥80,000/μL during the last 6 weeks of a 24-week treatment period. In some embodiments, at least 2 available weekly platelet counts are ≥85,000/μL during the last 6 weeks of a 24-week treatment period. In some embodiments, at least 2 available weekly platelet counts are ≥90,000/μL during the last 6 weeks of a 24-week treatment period. In some embodiments, at least 2 available weekly platelet counts are ≥95,000/μL during the last 6 weeks of a 24-week treatment period. In some embodiments, at least 2 available weekly platelet counts are ≥100,000/μL during the last 6 weeks of a 24-week treatment period. In some embodiments, at least 2 available weekly platelet counts are ≥250,000/μL during the last 6 weeks of a 24-week treatment period.

In some embodiments, the human patient has no two consecutive platelet counts ≤50,000/μL, wherein the platelet counts occur at least 4 weeks apart within a period of 24 weeks following at least one platelet count of ≥50,000/μL within 12 weeks of initiating rilzabrutinib treatment.

In some embodiments, the human patient has platelet counts of ≥50,000 for 4 out of the last 8 weeks of a 24-week treatment period. In some embodiments, the human patient has platelet counts of ≥55,000 for 4 out of the last 8 weeks of a 24-week treatment period. In some embodiments, the human patient has platelet counts of ≥60,000 for 4 out of the last 8 weeks of a 24-week treatment period. In some embodiments, the human patient has platelet counts of ≥65,000 for 4 out of the last 8 weeks of a 24-week treatment period. In some embodiments, the human patient has platelet counts of ≥70,000 for 4 out of the last 8 weeks of a 24-week treatment period. In some embodiments, the human patient has platelet counts of ≥75,000 for 4 out of the last 8 weeks of a 24-week treatment period. In some embodiments, the human patient has platelet counts of ≥80,000 for 4 out of the last 8 weeks of a 24-week treatment period. In some embodiments, the human patient has platelet counts of ≥85,000 for 4 out of the last 8 weeks of a 24-week treatment period. In some embodiments, the human patient has platelet counts of ≥90,000 for 4 out of the last 8 weeks of a 24-week treatment period. In some embodiments, the human patient has platelet counts of ≥95,000 for 4 out of the last 8 weeks of a 24-week treatment period. In some embodiments, the human patient has platelet counts of ≥100,000 for 4 out of the last 8 weeks of a 24-week treatment period. In some embodiments, the human patient has platelet counts of ≥250,000 for 4 out of the last 8 weeks of a 24-week treatment period.

In some embodiments, the human patient has platelet counts ≥50,000/μL on ≥4 of 6 biweekly platelet counts between weeks 14 and 24 of a 24-week treatment period. In some embodiments, the human patient has platelet counts ≥50,000/μL on 4 of 6 biweekly platelet counts between weeks 14 and 24 of a 24-week treatment period. In some embodiments, the human patient has platelet counts ≥50,000/μL on 5 of 6 biweekly platelet counts between weeks 14 and 24 of a 24-week treatment period. In some embodiments, the human patient has platelet counts ≥50,000/μL on 6 of 6 biweekly platelet counts between weeks 14 and 24 of a 24-week treatment period. In some embodiments, the human patient has platelet counts ≥55,000/μL. In some embodiments, the human patient has platelet counts ≥60,000/μL. In some embodiments, the human patient has platelet counts ≥65,000/μL. In some embodiments, the human patient has platelet counts ≥70,000/μL. In some embodiments, the human patient has platelet counts ≥75,000/μL. In some embodiments, the human patient has platelet counts ≥80,000/μL. In some embodiments, the human patient has platelet counts ≥85,000/μL. In some embodiments, the human patient has platelet counts ≥90,000/μL. In some embodiments, the human patient has platelet counts ≥95,000/μL. In some embodiments, the human patient has platelet counts ≥100,000/μL. In some embodiments, the human patient has platelet counts ≥250,000/μL.

In some embodiments, the human patient has platelet counts of ≥50,000 for two-thirds of ≥10 available weekly platelet counts during the last 16 weeks of a 53 week treatment period, further wherein: at least 3 available weekly platelet measurements are ≥50,000/μL during the last 8 weeks of the 53 week treatment period; and the human patient does not require rescue medication. In some embodiments, the human patient has platelet counts of ≥50,000 for 16 available weekly platelet counts during the last 16 weeks of a 53-week treatment period. of ≥50,000 for two-thirds of ≥10 available weekly platelet counts during the last 16 weeks of a 53-week treatment period. In some embodiments, the human patient has platelet counts ≥55,000/μL. In some embodiments, the human patient has platelet counts ≥60,000/μL. In some embodiments, the human patient has platelet counts ≥65,000/μL. In some embodiments, the human patient has platelet counts ≥70,000/μL. In some embodiments, the human patient has platelet counts ≥75,000/μL. In some embodiments, the human patient has platelet counts ≥80,000/μL. In some embodiments, the human patient has platelet counts ≥85,000/μL. In some embodiments, the human patient has platelet counts ≥90,000/μL. In some embodiments, the human patient has platelet counts ≥95,000/μL. In some embodiments, the human patient has platelet counts ≥100,000/μL. In some embodiments, the human patient has platelet counts ≥250,000/μL.

In some embodiments, wherein the human patient has platelet counts of ≥50,000 for two-thirds of ≥10 available weekly platelet counts during the last 16 weeks of a 53 week treatment period, and wherein: at least 3 available weekly platelet measurements are ≥50,000/μL during the last 8 weeks of the 53 week treatment period; and the human patient does not require rescue medication, the human patient does not require rescue medication during the last 8 weeks of the 53 week treatment period. In some embodiments, the human patient does not require rescue medication during the last 16 weeks of the 53 week treatment period. In some embodiments, the human patient does not require rescue medication during the 53 week treatment period.

In some embodiments, the human patient has: at least one platelet count ≥50,000/μL; or at least one platelet count between ≥30,000/μL and <50,000/μL and at least a doubling of the baseline platelet count during a 24-week treatment period in the absence of rescue medication. In some embodiments, the human patient has at least one platelet count ≥50,000/μL during a 24-week treatment period in the absence of rescue medication. In some embodiments, the human patient has at least one platelet count ≥50,000/μL and at least a doubling of the baseline platelet count during a 24-week treatment period in the absence of rescue medication. In some embodiments, the human patient has at least one platelet count between ≥30,000/μL and <50,000/μL and at least a doubling of the baseline platelet count during a 24-week treatment period in the absence of rescue medication.

In some embodiments, the human patient has at least one platelet count ≥100,000/μL during the treatment period, wherein the at least one platelet count occurs in the absence of bleeding. In some embodiments, the human patient has at least one platelet count ≥150,000/μL. In some embodiments, the human patient has at least one platelet count ≥200,000/μL.

In some embodiments, the human patient has at least 2 platelet counts of ≥100,000/μL on at least 2 consecutive platelet counts ≥5 days apart in the absence of rescue medication during the treatment period. In some embodiments, the human patient has at least one platelet count ≥150,000/μL. In some embodiments, the human patient has at least one platelet count ≥200,000/μL.

In some embodiments, the human patient has at least one platelet count of ≥250,000/μL during the treatment period. In some embodiments, the human patient has at least one platelet count of ≥300,000/μL. In some embodiments, the human patient has at least one platelet count of ≥350,000/μL. In some embodiments, the human patient has at least one platelet count of ≥400,000/μL.

In some embodiments, the human patient has at least one platelet count of ≥450,000/μL during the treatment period.

In some embodiments, the human patient has at least one platelet count ranging from 30,000/μL to 100,000/μL during the treatment period, wherein the at least one platelet count is at least double a baseline platelet count and occurs in the absence of bleeding. In some embodiments, the human patient has at least one platelet count ranging from 30,000/μL to 250,000/μL. In some embodiments, the human patient has at least one platelet count ranging from 30,000/μL to 450,000/μL In some embodiments, the human patient has at least one platelet count ranging from 30,000/μL to 100,000/μL, 35,000/μL to 100,000/μL, 40,000/μL to 100,000/μL, 45,000/μL to 100,000/μL, 50,000/μL to 100,000/μL, 55,000/μL to 100,000/μL, 60,000/μL to 100,000/μL, 65,000/μL to 100,000/μL, 70,000/μL to 100,000/μL, 75,000/μL to 100,000/μL, 80,000/μL to 100,000/μL, 85,000/μL to 100,000/μL, 90,000/μL to 100,000/μL, or 95,000/μL to 100,000/μL. In some embodiments, the human patient has at least one platelet count ranging from 30,000/μL to 35,000/μL, 30,000/μL to 40,000/μL, 30,000/μL to 45,000/μL, 30,000/μL to 50,000/μL, 30,000/μL to 55,000/μL, 30,000/μL to 60,000/μL, 30,000/μL to 65,000/μL, 30,000/μL to 70,000/μL, 30,000/μL to 75,000/μL, 30,000/μL to 80,000/μL, 30,000/μL to 85,000/μL, 30,000/μL to 90,000/μL, 30,000/μL to 95,000/μL, or 30,000/μL to 100,000/μL. In some embodiments, the human patient has at least one platelet count ranging from 30,000/μL to 35,000/μL, 35,000/μL to 40,000/μL, 40,000/μL to 45,000/μL, 45,000/μL to 50,000/μL, 50,000/μL to 55,000/μL, 55,000/μL to 60,000/μL, 60,000/μL to 65,000/μL, 65,000/μL to 70,000/μL, 70,000/μL to 75,000/μL, 75,000/μL to 80,000/μL, 80,000/μL to 85,000/μL, 85,000/μL to 90,000/μL, 90,000/μL to 95,000/μL, or 95,000/μL to 100,000/μL.

In some embodiments, the human patient has at least one platelet count ranging from ≥30,000/μL to <50,000/μL and at least a doubling of a baseline platelet count during the treatment period.

In some embodiments, the human patient receives concomitant treatment with TPO-RAs. In some embodiments, the human patient receives concomitant treatment with at least one TPO-RA selected from rTPO, romiplostim, eltrombopag, and avatrombopag. In some embodiments, the human patient receives concomitant treatment with rTPO. In some embodiments, the human patient receives concomitant treatment with romiplostim. In some embodiments, the human patient receives concomitant treatment with eltrombopag. In some embodiments, the human patient receives concomitant treatment with avatrombopag.

In some embodiments, the human patient has or has been identified as having a history of response to at least one prior line of therapy, wherein at least one prior therapy is chosen from a splenectomy, rituximab, TPO-RAs, intravenous immunoglobin (IVIG), corticosteroids, anti-D immunoglobulin therapy, and immunosuppressive drugs. In some embodiments, the human patient has a history of response to a splenectomy. In some embodiments, the human patient has a history of response to rituximab. In some embodiments, the human patient has a history of response to TPO-RAs. In some embodiments, the human patient has a history of response to intravenous immunoglobin (IVIG). In some embodiments, the human patient has a history of response to corticosteroids. In some embodiments, the patient has a history of response to at least one corticosteroid selected from dexamethasone or oral prednisone/prednisolone. In some embodiments, the patient has a history of response to dexamethasone. In some embodiments, the patient has a history of response to oral prednisone/prednisolone. In some embodiments, the human patient has a history of response to anti-D immunoglobulin therapy. In some embodiments, the human patient has a history of response to immunosuppressive drugs. In some embodiments, the human patient has a history of response to at least one immunosuppressive drug selected from fostamatinib, mycophenolate mofetil (MMF), and cyclosporine prior to the start of the treatment period. In some embodiments, the human patient has a history of response to fostamatinib. In some embodiments, the human patient has a history of response to mycophenolate mofetil (MMF). In some embodiments, the human patient has a history of response to cyclosporine.

In some embodiments, the human patient does not require rescue medication during the treatment period.

In some embodiments, the human patient has or has been identified as having had a splenectomy prior to the start of treatment.

In some embodiments, the human patient has or has been identified as having a history of taking rituximab prior to the start of the treatment period. In some embodiments, the human patient has not received prior treatment with rituximab.

In some embodiments, the human patient has or has been identified as having a history of taking at least one TPO-RA prior to the start of the treatment period. In some embodiments, the human patient has or has been identified as having a history of taking at least one TPO-RA selected from rTPO, romiplostim, eltrombopag, and avatrombopag prior to the start of the treatment period. In some embodiments, the human patient has or has been identified as having a history of taking rTPO. In some embodiments, the human patient has or has been identified as having a history of taking romiplostim. In some embodiments, the human patient has or has been identified as having a history of taking eltrombopag. In some embodiments, the human patient has or has been identified as having a history of taking avatrombopag. In some embodiments, the human patient has not received prior treatment with one or more TPO-RAs.

In some embodiments, the human patient has or has been identified as having a history of taking intravenous immunoglobin (IVIG) prior to the start of the treatment period.

In some embodiments, the human patient has or has been identified as having a history of taking at least one corticosteroid prior to the start of the treatment period. In some embodiments, the human patient has or has been identified as having a history of taking at least one corticosteroid selected from dexamethasone or oral prednisone/prednisolone prior to the start of the treatment period. In some embodiments, the human patient has or has been identified as having a history of taking dexamethasone. In some embodiments, the human patient has or has been identified as having a history of taking oral prednisone/prednisolone.

In some embodiments, the human patient has or has been identified as having a history of taking anti-D immunoglobulin therapy prior to the start of the treatment period.

In some embodiments, the human patient has or has been identified as having a history of taking at least one immunosuppressive drug prior to the start of the treatment period. In some embodiments, the human patient has or has been identified as having a history of taking at least one immunosuppressive drug selected from fostamatinib, mycophenolate mofetil (MMF), and cyclosporine prior to the start of the treatment period. In some embodiments, the human patient has or has been identified as having a history of taking fostamatinib. In some embodiments, the human patient has or has been identified as having a history of taking mycophenolate mofetil (MMF). In some embodiments, the human patient has or has been identified as having a history of taking cyclosporine.

In some embodiments, the human patient has or has been identified as having a response to the prior ITP therapy.

In some embodiments, the response to the prior ITP therapy comprised a platelet count of <50,000/μL. In some embodiments, the response to the prior ITP therapy comprised a platelet count of <45,000/μL. In some embodiments, the response to the prior ITP therapy comprised a platelet count of <40,000/μL. In some embodiments, the response to the prior ITP therapy comprised a platelet count of <35,000/μL. In some embodiments, the response to the prior ITP therapy comprised a platelet count of <30,000/μL. In some embodiments, the response to the prior ITP therapy comprised a platelet count of <25,000/μL. In some embodiments, the response to the prior ITP therapy comprised a platelet count of <20,000/μL. In some embodiments, the response to the prior ITP therapy comprised a platelet count of <15,000/μL. In some embodiments, the response to the prior ITP therapy comprised a platelet count of <10,000/μL. In some embodiments, the response to the prior ITP therapy comprised a platelet count of <5,000/μL. In some embodiments, the response to the prior ITP therapy comprised a platelet count of <4,000/μL. In some embodiments, the response to the prior ITP therapy comprised a platelet count of <3,000/μL. In some embodiments, the response to the prior ITP therapy comprised a platelet count of <2,000/μL. In some embodiments, the response to the prior ITP therapy comprised a platelet count of <1,000/μL.

In some embodiments, the response to the prior ITP therapy was not sustained.

In some embodiments, the patient has or has been identified as having a documented intolerance to standard-of-care ITP therapies.

In some embodiments, the patient has or has been identified as having a contraindication for standard-of-care ITP therapies.

In some embodiments, the human patient has primary ITP.

In some embodiments, the human patient is aged ≥18 years. In some embodiments, the human patient is an adult.

In some embodiments, the human patient is aged <18 years. In some embodiments, the human patient is a pediatric patient.

In some embodiments, the human patient is aged 12 to <18 years. In some embodiments, the human patient is an adolescent.

In some embodiments, the human patient is aged <12 years. In some embodiments, the human patient is aged 10 to <12 years. In some embodiments, the human patient is a child.

In some embodiments, the human patient is aged 10 to <18 years.

In some embodiments, the human patient has or has been identified as having had ITP for a duration of >1 year. In some embodiments, the human patient has or has been identified as having had ITP for a duration of >2 years. In some embodiments, the human patient has or has been identified as having had ITP for a duration of >3 years. In some embodiments, the human patient has or has been identified as having had ITP for a duration of >4 years. In some embodiments, the human patient has or has been identified as having had ITP for a duration of >5 years. In some embodiments, the human patient has or has been identified as having had ITP for a duration of >10 years. In some embodiments, the human patient has or has been identified as having had ITP for a duration of >15 years. In some embodiments, the human patient has or has been identified as having had ITP for a duration of >20 years. In some embodiments, the human patient has or has been identified as having had ITP for a duration of >25 years. In some embodiments, the human patient has or has been identified as having had ITP for a duration of >30 years. In some embodiments, the human patient has or has been identified as having had ITP for a duration of >35 years. In some embodiments, the human patient has or has been identified as having had ITP for a duration of >40 years. In some embodiments, the human patient has or has been identified as having had ITP for a duration of >45 years. In some embodiments, the human patient has or has been identified as having had ITP for a duration of >50 years.

In some embodiments, the human patient does not have chronic ITP.

In some embodiments, the human patient has persistent ITP.

In some embodiments, the human patient has chronic ITP.

In some embodiments, the human patient has relapsing ITP.

In some embodiments, the human patient has refractory ITP.

In some embodiments, the treatment period is at least 168 days. In some embodiments, the treatment period is at least 364 days. In some embodiments, the treatment period is at least 392 days. In some embodiments, the treatment period is at least 728 days.

In some embodiments, the method comprises administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day. In some embodiments, the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

In some embodiments, the method comprises administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day. In some embodiments, the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

In some embodiments, the at least one compound is orally administered to the human patient. In some embodiments, the at least one compound is administered to the human patient in the form of at least one tablet. In some embodiments, the at least one compound is administered with water.

In some embodiments, the patient is ≥18 years of age. In some embodiments, the patient is an adult.

In some embodiments, the patient is <18 years of age. In some embodiments, the patient is a pediatric patient.

In some embodiments, the patient is 12 to <18 years of age. In some embodiments, the patient is an adolescent.

In some embodiments, the patient is 10 to <12 years of age. In some embodiments, the patient is a child.

In some embodiments, the patient experiences a reduction in fatigue. In some embodiments, wherein the patient experiences a reduction in fatigue, the human patient experiences a reduction in fatigue within 1 week of initiating treatment. In some embodiments, the human patient experiences a reduction in fatigue within 2 weeks of initiating treatment. In some embodiments, the human patient experiences a reduction in fatigue within 3 weeks of initiating treatment. In some embodiments, the human patient experiences a reduction in fatigue within 4 weeks of initiating treatment. In some embodiments, the human patient experiences a reduction in fatigue within 5 weeks of initiating treatment. In some embodiments, the human patient experiences a reduction in fatigue within 6 weeks of initiating treatment. In some embodiments, the human patient experiences a reduction in fatigue within 7 weeks of initiating treatment. In some embodiments, the human patient experiences a reduction in fatigue within 8 weeks of initiating treatment. In some embodiments, the human patient experiences a reduction in fatigue within 9 weeks of initiating treatment. In some embodiments, the human patient experiences a reduction in fatigue within 10 weeks of initiating treatment. In some embodiments, the human patient experiences a reduction in fatigue within 11 weeks of initiating treatment. In some embodiments, the human patient experiences a reduction in fatigue within 12 weeks of initiating treatment. In some embodiments, the human patient experiences a reduction in fatigue within 13 weeks of initiating treatment. In some embodiments, the human patient experiences a reduction in fatigue within 14 weeks of initiating treatment. In some embodiments, the human patient experiences a reduction in fatigue within 15 weeks of initiating treatment. In some embodiments, the human patient experiences a reduction in fatigue within 16 weeks of initiating treatment. In some embodiments, the human patient experiences a reduction in fatigue within 17 weeks of initiating treatment. In some embodiments, the human patient experiences a reduction in fatigue within 18 weeks of initiating treatment. In some embodiments, the human patient experiences a reduction in fatigue within 19 weeks of initiating treatment. In some embodiments, the human patient experiences a reduction in fatigue within 20 weeks of initiating treatment. In some embodiments, the human patient experiences a reduction in fatigue within 21 weeks of initiating treatment. In some embodiments, the human patient experiences a reduction in fatigue within 22 weeks of initiating treatment. In some embodiments, the human patient experiences a reduction in fatigue within 23 weeks of initiating treatment. In some embodiments, the human patient experiences a reduction in fatigue within 24 weeks of initiating treatment. In some embodiments, the human patient experiences a reduction in fatigue within 53 weeks of initiating treatment. In some embodiments, the human patient experiences a reduction in fatigue as determined using ITP Patient Assessment Questionnaire™ (ITP-PAQ™). In some embodiments, the human patient experiences a reduction in fatigue as determined using the ITP Kids' ITP Tools (ITP-KIT).

In some embodiments, the human patient experiences a change from baseline in IBLS at weeks 13 and 15 of a treatment period. In some embodiments, the human patient experiences a change from baseline in IBLS at week 25 of a treatment period.

In some embodiments, the human patient experiences an improvement in quality of life. In some embodiments, the human patient experiences an improvement in quality of life as determined by the ITP-KIT.

In some embodiments, the human patient experiences a change from baseline on the Symptoms, Bother-Physical Health, Activity, Fatigue/Sleep, Psychological Health, Fear, Social Activity, Women's Reproductive Health, and Work domains of the ITP-PAQ™ In some embodiments, the human patient experiences a change from baseline on the Symptoms domain of the ITP-PAQ™. In some embodiments, the human patient experiences a change from baseline on the Bother-Physical Health domain of the ITP-PAQ™. In some embodiments, the human patient experiences a change from baseline on the Activity domain of the ITP-PAQ™. In some embodiments, the human patient experiences a change from baseline on the Fatigue/Sleep domain of the ITP-PAQ™. In some embodiments, the human patient experiences a change from baseline on the Psychological Health domain of the ITP-PAQ™. In some embodiments, the human patient experiences a change from baseline on the Fear domain of the ITP-PAQ™. In some embodiments, the human patient experiences a change from baseline on the Social Activity domain of the ITP-PAQ™. In some embodiments, the human patient experiences a change from baseline on the Women's Reproductive Health domain of the ITP-PAQ™. In some embodiments, the human patient experiences a change from baseline on the Work domain of the ITP-PAQ™.

In some embodiments, the human patient experiences less frequent or less severe bleeding than a human patient administered a therapeutically effective amount of another BTK inhibitor.

In some embodiments, the human patient has or has been identified as having become refractory to at least one prior line of therapy, for example at least two, for example at least three, for example at least four, for example at least five, for example at least six, for example at least seven, for example at least eight, for example at least nine, for example at least 10, for example at least 11, for example at least 12, for example at least 13, for example at least 14, for example at least 15, for example at least 16, for example at least 17 prior lines of therapy. In some embodiments, the patient has or has been identified as having become refractory to at least one prior line of therapy. In some embodiments, the patient has or has been identified as having become refractory to at least two prior lines of therapy. In some embodiments, the patient has or has been identified as having become refractory to at least three prior lines of therapy. In some embodiments, the patient has or has been identified as having become refractory to at least four prior lines of therapy. In some embodiments, the patient has or has been identified as having become refractory to at least five prior lines of therapy. In some embodiments, the patient has or has been identified as having become refractory to at least six prior lines of therapy. In some embodiments, the patient has or has been identified as having become refractory to at least seven prior lines of therapy. In some embodiments, the patient has or has been identified as having become refractory to at least eight prior lines of therapy. In some embodiments, the patient has or has been identified as having become refractory to at least nine prior lines of therapy. In some embodiments, the patient has or has been identified as having become refractory to at least 10 prior lines of therapy. In some embodiments, the patient has or has been identified as having become refractory to at least 11 prior lines of therapy. In some embodiments, the patient has or has been identified as having become refractory to at least 12 prior lines of therapy. In some embodiments, the patient has or has been identified as having become refractory to at least 13 prior lines of therapy. In some embodiments, the patient has or has been identified as having become refractory to at least 14 prior lines of therapy. In some embodiments, the patient has or has been identified as having become refractory to at least 15 prior lines of therapy. In some embodiments, the patient has or has been identified as having become refractory to at least 16 prior lines of therapy. In some embodiments, the patient has or has been identified as having become refractory to at least 17 prior lines of therapy.

Some embodiments of the present disclosure relate to a method of treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient has or has been identified as having an insufficient response or intolerance to one or more previous treatments.

Some embodiments of the present disclosure relate to the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for treating immune thrombocytopenia (ITP) in a human patient with persistent or chronic ITP in need thereof.

Some embodiments of the present disclosure relate to a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for use as a medicament for treating immune thrombocytopenia (ITP) in a human patient with persistent or chronic ITP in need thereof.

Some embodiments of the present disclosure relate to a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for use for treating immune thrombocytopenia (ITP) in a human patient with persistent or chronic ITP in need thereof.

Some embodiments of the present disclosure relate to the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, as a medicament for treating immune thrombocytopenia (ITP) in a human patient with persistent or chronic ITP in need thereof.

### Pharmaceutical Compositions:

In some embodiments of the present disclosure, rilzabrutinib is administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet.

In some embodiments of the present disclosure, rilzabrutinib is orally administered as part of a pharmaceutical composition comprising: at least one compound chosen from rilzabrutinib and pharmaceutically acceptable salts thereof, and at least one pharmaceutically acceptable excipient. In some embodiments, the pharmaceutical composition is in the form of at least one tablet.

In some embodiments, rilzabrutinib is administered in the form of a film-coated tablet.

In some embodiments of the present disclosure, rilzabrutinib is administered in the form of at least one tablet comprising: at least one compound chosen from rilzabrutinib and pharmaceutically acceptable salts thereof, and at least one pharmaceutically acceptable excipient. In some embodiments, rilzabrutinib is administered in the form of at least one tablet comprising: at least one compound chosen from rilzabrutinib and pharmaceutically acceptable salts thereof; at least one filler; at least one disintegrant; at least one lubricant; and at least one film coating.

In some embodiments, rilzabrutinib is administered with a glass of water.

The proportion and nature of any pharmaceutically acceptable excipient may be determined by the chosen route of administration and standard pharmaceutical practice. Except insofar as any conventional pharmaceutically acceptable excipient is incompatible with rilzabrutinib, such as by producing any undesirable biological effect or otherwise interacting in a deleterious manner with any other component(s) of the pharmaceutically composition, its use is contemplated to be within the scope of this disclosure.

Some non-limiting examples of materials which may serve as pharmaceutically acceptable excipients include: (1) sugars, such as, e.g., lactose, glucose, and sucrose; (2) starches, such as, e.g., corn starch and potato starch; (3) cellulose and its derivatives, such as, e.g., sodium carboxymethyl cellulose, ethyl cellulose, and cellulose acetate; (4) powdered tragacanth; (5) malt; (6) gelatin; (7) talc; (8) excipients, such as, e.g., cocoa butter and suppository waxes; (9) oils, such as, e.g., peanut oil, cottonseed oil, safflower oil, sesame oil, olive oil, corn oil, and soybean oil; (10) glycols, such as, e.g., propylene glycol; (11) polyols, such as, e.g., glycerin, sorbitol, mannitol, and polyethylene glycol; (12) esters, such as, e.g., ethyl oleate and ethyl laurate; (13) agar; (14) buffering agents, such as, e.g., magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York also discloses additional non-limiting examples of pharmaceutically acceptable excipients, as well as known techniques for preparing and using the same.

One skilled in the art can readily select the proper form and route of administration depending upon the disorder or condition to be treated, the stage of the disorder or condition, and other relevant circumstances.

### EXAMPLES

The following example is intended to be illustrative and is not meant in any way to limit the scope of the disclosure.

### ABBREVIATIONS

API Active pharmaceutical ingredient
ALT Alanine aminotransferase
AST Aspartate aminotransferase
bid/BID Twice daily (morning and evening)
BTK Bruton's Tyrosine Kinase
CS Corticosteroids
ECG Electrocardiogram
EQ-5D-5L Euro-QoL 5-Dimension 5 Level
HRQOL Health-related quality of life
IBLS ITP Bleeding Scale
ICH International Conference on Harmonization
ICMJE International Committee of Medical Journal Editors
IgG Immunoglobulin G
ITP Immune thrombocytopenic purpura
ITP-KIT ITP Kids' ITP Tools
ITP-PAQ™ ITP Patient Assessment Questionnaire™
IV Intravenous
IVIG Intravenous immunoglobulin
IWG International Working Group
LTE Long term extension
MID Minimal important difference
NE Not estimable
PO Oral
qd/QD Once a day
Q2d/Q2D Every other day
QOL Quality of Life
QTc QT interval corrected for heart rate
QTcF QT interval corrected by Fridericia
rTPO Recombinant thrombopoietin
SI Système international d'unités (International system of units)
TPO Thrombopoietin
TPO-RA Thrombopoietin receptor agonist
ULN Upper limit of normal
VAS Visual Analog Scale
WHODD World Health Organization Drug Dictionary

### Example 1: A Phase 3, Multicenter, Randomized, Double-Blind, Placebo-Controlled, Parallel-Group Study with an Open-Label Extension to Evaluate the Efficacy and Safety of Oral Rilzabrutinib (PRN1008) in Adults, Adolescents, and Children with Persistent or Chronic Immune Thrombocytopenia (ITP) (LUNA 3)

### Study Overview

The LUNA 3 trial is an ongoing, multicenter, randomized, double-blind, placebo-controlled, parallel group, interventional phase 3 study evaluating the efficacy and safety of oral rilzabrutinib in adult and pediatric patients with persistent or chronic ITP (Study to evaluate rilzabrutinib in adults, adolescents, and children with persistent or chronic immune thrombocytopenia (ITP), A phase 3, multicenter, randomized, double-blind, placebo-controlled, parallel-group study with an open-label extension to evaluate the efficacy and safety of oral rilzabrutinib (PRN1008) in adults, adolescents, and children with persistent or chronic immune thrombocytopenia (ITP)) LUNA 3 has unique design features to include both 194 adult and 30 pediatric patients.

### Study Rationale

The LUNA 3 phase 3 trial will compare the magnitude, durability, and stability of rilzabrutinib's efficacy and safety (as compared with the placebo) in adults with persistent or chronic ITP and explore findings in pediatric patients. In this trial, a durable response (the primary endpoint) is defined as (1) the achievement of platelet counts of ≥50×10⁹/μL for at least two-thirds of ≥8 available, weekly scheduled platelet measurements during the last 12 weeks of the 24-week blinded treatment period in the absence of rescue therapy (e.g., in 8 of 12 or 6 of 9 counts) and provided that at least 2 available weekly scheduled platelet measurements are at or above 50,000/μL during the last 6 weeks of the 24-week blinded treatment period; or (2) the achievement of platelet counts of ≥50×10⁹/μL for ≥8 out of the 12 scheduled observations in the last 12 weeks of the 24-week double-blinded treatment period in the absence of rescue therapy (EU and UK).

The International Working Group (IWG) defines a platelet response as the achievement of any platelet count between 30×10⁹/μL to 100×10⁹/μL and at least doubling from the baseline count. A complete response is defined as any platelet count ≥100×10⁹/μL; both endpoints require the absence of bleeding (Rodeghiero et al. 2009). Although a lower platelet level is sufficient to establish a platelet response as defined by the IWG guidelines, clinical trials of approved ITP therapies commonly use a count of ≥50×10⁹/μL as a threshold for establishing a platelet response (Bussel et al. 2018, Ghanima et al. 2015, Jurczak et al. 2018, Kuter et al. 2008).

A durable platelet response, defined as platelet counts ≥30×10⁹/μL and at least doubling from the baseline at 6 months, is a goal for ITP therapies as an endpoint that measures clinical benefit over time (Neunert et al. 2019). For the recently approved drug fostamatinib, durable response was demonstrated when two-thirds of platelet counts during the last 12 weeks (6 visits) of the blinded period met the primary endpoint: platelet counts ≥50×10⁹/μL on ≥4 of the 6 biweekly visits (weeks 14, 16, 18, 20, 22, and 24) (Bussel J et al. 2018, Bussel J et al. 2019). This definition of durable response was based on the proportion of available platelet counts ≥50×10⁹/μL instead of the fixed timepoint of platelet counts ≥30×10⁹/μL and doubled from baseline at 6 months as defined in guidelines, potentially allowing missing platelet counts to be taken into account (Neunert et al. 2019). A significant number of both adults, adolescents, and children with ITP experience fatigue is thought to be associated with low platelets (<30×10⁹/μL) and caused by a chronic inflammatory state and immune dysregulation (Trotter and Hill 2018, Hill Q A and Newland A C 2015, Newton et al. 2011). To date, no available ITP therapies showed significant improvement in fatigue in a randomized placebo-controlled clinical trial report. ITP Patient Assessment Questionnaire (ITP-PAQ™) item 10 (Fatigue/Sleep domain) is being employed to quantify the impact of treatment with rilzabrutinib on fatigue as a key secondary endpoint.

Patients enrolled in this trial are expected to have previously responded to at least one prior ITP therapy, supporting the diagnosis of primary ITP, which is a diagnosis by exclusion (negative diagnosis); preliminary findings based on phase 2 study results suggest that patients naive to rituximab and TPO-RA may achieve a better response rate (Kuter et al. 2022).

### Patient Eligibility and Recruitment

Eligible patients must have primary ITP (Provan et al. 2019) with a duration of >3 months if aged ≥18 years or a duration of >6 months if aged 10 to <12 years (EU countries only) or 12 to <18 years (all countries). Patients should have an initial platelet count of <30×10⁹/μL, with no single platelet count >35×10⁹/μL within 14 days before the first dose of rilzabrutinib. Additionally, patients should have had a previous response (platelet counts ≥50×10⁹/μL) to CS or IVIG/anti-D. The prior response was not sustained; alternatively, the patient must have a documented intolerance, history of insufficient response, or contraindication to any appropriate courses of standard ITP therapy. Patients on a stable dose of CS and/or TPO-RA are eligible to participate. Adequate hematologic, hepatic, and renal function (absolute neutrophil count ≥1.5×10⁹/μL, aspartate aminotransferase/alanine aminotransferase 1.5× upper limit of normal [ULN], albumin ≥3 g/dL, total bilirubin ≤15× ULN [unless documented Gilbert syndrome], estimated glomerular filtration rate >50 by Cockcroft and Gault method) and hemoglobin levels >9 g/dL (within 1 week before study day 1) are required. Additional eligibility criteria are outlined in Table 1.

TABLE 1
Key Eligibility Criteria.

| Inclusion Criteria | Exclusion Criteria |
|---|---|
| Primary ITP for >3 months if aged ≥18 | Secondary ITP |
| years or >6 months if aged 12 to <18 | Pregnant or lactating women |
| years | Electrocardiogram (ECG) findings |
| In EU countries, pediatric patients | Aged ≥10 and <16 years: QTcF >449 |
| aged 10-18 years are eligible | msec (males) or >457 msec (females) |
| Previous response (platelet count ≥50 × 10⁹/L) | Aged ≥16 and <18 years: QTcF >450 |
| to CS or IVIG/anti-D that | msec (males) or >460 msec (females) |
| was not sustained, or documented | Aged ≥18 years, of QTcF >450 msec |
| intolerance, insufficient response, or | (males) or >470 msec (females), poorly |
| contraindication to any appropriate | controlled atrial fibrillation (i.e., |
| courses of standard-of-care ITP therapy | symptomatic participants or a |
| An initial platelet count (the average of | ventricular rate >100 beats/min on |
| 2 platelet counts ≥5 days apart during | ECG), or other clinically significant |
| screening) of <30 × 10⁹/L and no single | abnormalities |
| platelet count >35 × 10⁹/L within 2 | Transfusions or use of any other rescue |
| weeks prior to study treatment | medications with intent to increase |
| If aged <18 years, must require ITP | platelet count within 2 weeks prior to |
| treatment per clinical investigator's | study treatment |
| assessment | Change in CS and/or TPO-RA dose |
| Children aged 10 to <12 years must | within 2 weeks prior to study treatment |
| have a body weight of ≥30 kg | (>10% variation from current doses) |
| Adequate hematologic, hepatic, and | Use of immunosuppressants other than |
| renal function (absolute neutrophil | CS within 5 times the elimination half- |
| count ≥1.5 × 10⁹/L, AST/ALT ≤1.5 × | life of the drug or within 2 weeks prior |
| upper limit of normal (ULN), albumin ≥3 g/dL, | to study treatment, whichever is longer |
| total bilirubin ≤1.5 × ULN | Treatment with rituximab or |
| (unless the patient has documented | splenectomy within 3 months prior to |
| Gilbert syndrome), glomerular filtration | study treatment (patients previously |
| rate >50 (Cockcroft and Gault method)) | treated with rituximab must have |
| Hemoglobin >9 g/dL within 1 week | normal B cell counts prior to |
| prior to study treatment | enrollment) |
| | Prior rilzabrutinib therapy |
| | Treatment with any investigational drug |
| | within 5 times the elimination half-life |
| | of the drug or within the 30 days prior |
| | to study treatment, whichever is longer; |
| | use of an investigational drug at the |
| | time of dosing |
| | History (within 5 years of study day 1) |
| | of current active malignancy requiring |
| | or likely to require chemotherapeutic or |
| | surgical treatment during the study, |
| | except for non-melanoma skin cancer |
| | History of solid organ transplant or |
| | planned surgery |
| | Myelodysplastic syndrome |
| | COVID-19 vaccine within 2 weeks |
| | prior or planned during last 12 weeks |
| | of blinded treatment |
| | Live vaccine within 28 days prior to |
| | study treatment or during the study |
| | Planned or concomitant use of any |
| | anticoagulants and platelet aggregation |
| | inhibiting drugs such as aspirin (except |
| | for low dose aspirin up to 100 mg per |
| | day), nonsteroidal anti-inflammatory |
| | drugs, and/or thienopyridines within 14 |
| | days of treatment initiation and until the |
| | end of the active treatment period |
| | Any other clinically significant disease, |
| | condition, known allergy to any of the |
| | study medications, their analogs, or |
| | excipients in the various formulations |
| | of any agent, or medical history that, in |
| | the opinion of the investigator or |
| | sponsor's medical monitor, would |
| | interfere with participant safety, study |
| | evaluations, and/or study procedures |

Patients with known secondary ITP are not eligible to enroll. Key exclusion criteria also include platelet transfusions or use of any other rescue medications (e.g., IVIG) with the intent to increase platelet counts, changes in CS and/or TPO-RA dose (>10% variation from current doses) within 2 weeks before study entry, and receipt of a live vaccine within 28 days before study day 1 (or the intention to receive a live vaccine during the study). Administration of a COVID-19 vaccine specifically within 2 weeks prior to study treatment and during the last 12 weeks of the blinded treatment period is not allowed due to potential confounding effects on the primary endpoint (Kuter D J 2021, Lee et al. 2022).

### Study Design and Interventions

For each patient, the study will last up to 60 weeks from the start of the screening period to the end of the study visit. After providing written informed consent, patients enter a 28-day screening period and eligible patients are randomized in a 2:1 ratio to receive oral treatment with either rilzabrutinib 400 mg BID or placebo with optional stable doses of standard CS and/or TPO-RA therapy.

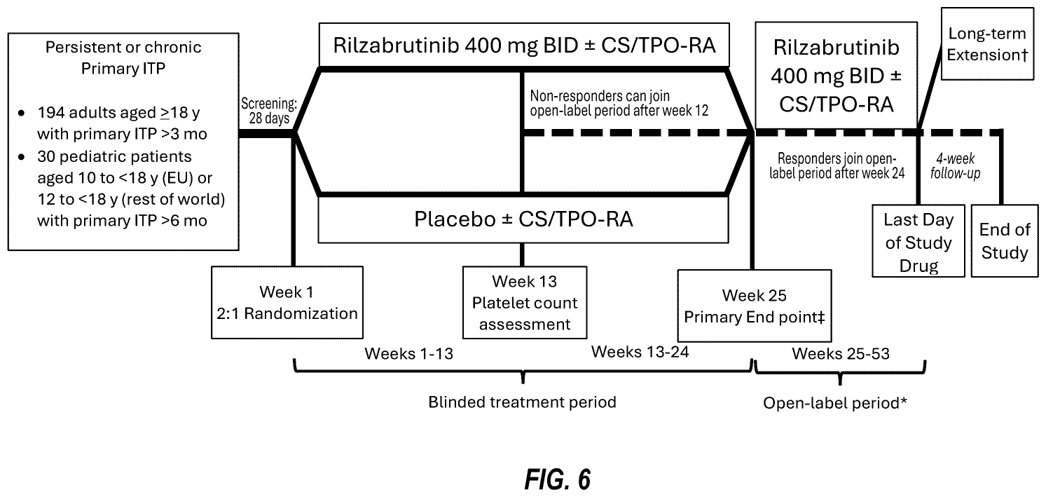

Randomization is stratified by splenectomy status (yes/no) and severity of disease (platelet counts <15×10⁹/μL vs ≥1 5×10⁹/μL) and is carried out separately for adult and pediatric patients. After randomization, patients start a blinded treatment period for up to 24 weeks, followed by a 28-week open-label period when all patients receive rilzabrutinib, and then a 4-week safety follow-up or long-term extension (LTE) phase (FIG. 6). Stable doses of concomitant ITP medication (oral CS and/or TPO-RA treatment) are permitted in both treatment arms, with dose reduction allowed. CS and/or TPO-RA administration should follow the corresponding current package inserts and/or the summary of product characteristics of the country-specific marketing authorization.

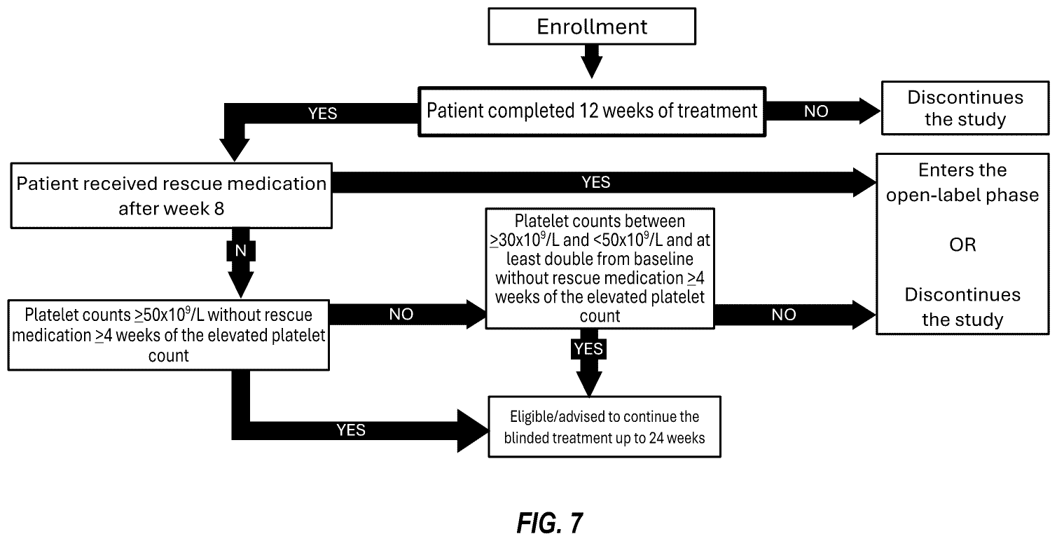

At the end of 12 weeks of treatment with rilzabrutinib 400 mg BID or a placebo, patients are assessed for achieving a platelet response, which is defined as a platelet count of ≥50×10⁹/μL or between ≥30×10⁹/μL and <50×10⁹/μL and at least doubled from baseline at any time and in the absence of rescue medication in the 4 weeks prior to the elevated platelet count that meets the platelet response criteria (FIG. 7). Baseline is defined as the mean of 2 qualifying platelet counts at screening and the day 1 platelet count before the first dose of rilzabrutinib or placebo. Next, responders continue the blinded treatment period for 12 more weeks (for a total of 24 weeks) before entering the 28-week open-label period, whereas non-responders may discontinue from the study or enter the 28-week open-label period at the end of week 12 while receiving rilzabrutinib 400 mg BID. Irrespective of the non-responder's choice, initial study medication assignment will remain blinded.

Patients are assessed for eligibility to enter the LTE at the end of the open-label period (i.e., after 28 weeks of treatment with rilzabrutinib 400 mg BID). After completing the open-label period, any participants who demonstrate a platelet response defined as platelet counts ≥50×10⁹/μL or ≥30×10⁹/μL and at least doubled from baseline at ≥50% of the visits without receiving rescue therapy while on rilzabrutinib treatment during the last 8 weeks of the open-label period will be allowed to enter the LTE. In the LTE part, concomitant CS and/or TPO-RA dose reductions or discontinuations are allowed if platelet counts are ≥50×10⁹/μL on 3 scheduled visits over 12 weeks. Patients who enter the LTE will continue to receive treatment until the time expected for the last participant who enters the LTE to complete 12 months.

In the first study phase, which will span 24 weeks, treatments are double-blinded to the investigator/clinical study team and patients, unless unblinding is deemed medically necessary by the investigator after consulting with the study sponsor's medical monitor and in accordance with local regulations/policies. The treating study investigator will be responsible for ensuring treatment is administered in compliance with the study protocol. Patients may discontinue the trial temporarily due to suspected adverse events or a regional/national emergency declared by a governmental agency (e.g., a pandemic); reinitiate treatment under close supervision of the study investigator if continued eligibility criteria are met; or withdraw from the study due to life-threatening or grade 4 treatment-related adverse events, serious allergic reaction, pregnancy, any medical condition/personal circumstance deemed by the investigator to pose a significant risk to the patient, human immunodeficiency or hepatitis B/C viral infections, protocol violation compromising data interpretation, or abnormal liver tests.

### Endpoints and Assessments

The primary endpoint of this study is durable platelet response, defined as (1) the achievement of platelet counts of ≥50×10⁹/μL for at least two-thirds of ≥8 available weekly scheduled platelet measurements during the last 12 weeks of the 24-week blinded treatment period in the absence of rescue therapy, provided that ≥2 available weekly scheduled platelet measurements are ≥50×10⁹/μL during the last 6 weeks of the 24-week blinded treatment period; or (2) the achievement of platelet counts of ≥50×10⁹/μL for ≥8 out of the 12 scheduled observations in the last 12 weeks of the 24-week blinded treatment period in the absence of rescue medication (EU and UK) (Table 2). Key secondary efficacy endpoints are the number of weeks with platelet counts ≥50×10⁹/μL or between ≥30×10⁹/μL and <50×10⁹/μL and at least doubled from baseline during the 24-week blinded treatment period in the absence of rescue medication, the number of weeks with platelet counts ≥30×10⁹/μL and at least doubled from baseline during the 24-week blinded treatment period in the absence of rescue medication, time to first platelet counts ≥50×10⁹/μL or between ≥30×10⁹/μL and <50×10⁹/μL and at least doubled from baseline, the proportion of patients requiring rescue therapy during the 24-week blinded treatment period, and changes from baseline on ITP Patient Assessment Questionnaire™ (ITP-PAQ™) physical fatigue score in adult patients (≥18 years of age) at week 13 of treatment.

TABLE 2
LUNA 3 Study Endpoints.

| | |
|---|---|
| Primary | (1) Platelet counts ≥50 × 10⁹/L for ≥two-thirds of ≥8 available |
| Endpoint | weekly scheduled platelet measurements during the last 12 weeks |
| | of the 24-week blinded treatment period in the absence of rescue |
| | medication, provided that at least 2 available weekly scheduled |
| | platelet measurements are ≥50 × 10⁹/L during the last 6 weeks |
| | of the 24-week blinded treatment period; or (2) platelet counts |
| | of ≥50 × 10⁹/L for ≥8 out of the 12 scheduled observations in the |
| | last 12 weeks of the 24-week blinded treatment period in the absence |
| | of rescue medication (EU and UK) |
| Key Secondary | (1) Number of weeks with platelet counts ≥50 × 10⁹/L or (2) |
| Endpoints | number of weeks with platelet counts between ≥30 × 10⁹/L |
| | and <50 × 10⁹/L and at least doubled from baseline during the |
| | 24-week blinded treatment period in the absence of rescue medication |
| | Number of weeks with platelet counts ≥30 × 10⁹/L and at least |
| | doubled from baseline during the 24-week blinded treatment |
| | period in the absence of rescue medication |
| | (1) Time to first platelet count ≥50 × 10⁹/L or (2) time to first |
| | platelet count between ≥30 × 10⁹/L and <50 × 10⁹/L and at least |
| | doubled from baseline |
| | Proportion of patients requiring rescue therapy during the 24- |
| | week blinded treatment period |
| | Changes from baseline on ITP-PAQ ™ physical fatigue score in |
| | adult patients (≥18 years of age) at Week 13 of treatment |
| | Safety |
| | Change from baseline in IBLS at Week 25 (EU (EEA countries) |
| | and UK-specific requirements) |
| Other | Stability of response, defined as the proportion of participants |
| Secondary | able to achieve a stable platelet response, which is defined as the |
| Endpoints: | proportion of patients with no 2 scheduled visits, at least 4 weeks |
| Efficacy | apart, with a platelet count <50 × 10⁹/L, without an intervening |
| Endpoint | visit with a platelet count ≥50 × 10⁹/L, within a period of 24 weeks |
| | following initial achievement of the platelet response. |
| Other | Frequency and severity of TEAEs |
| Secondary | Frequency and severity of bleeding TEAEs |
| Endpoints: | Change from baseline in physical examination, ECG, vital signs, |
| Safety | and clinical laboratory test results (serum chemistry and |
| Endpoints | hematology (except for platelet counts included in the primary |
| | efficacy endpoint)) |
| Other | Plasma concentration of rilzabrutinib |
| Secondary | |
| Endpoints: | |
| Pharmacokinetic | |
| Endpoints | |
| Other | Change from baseline on the Symptoms, Bother-Physical Health, |
| Secondary | and Activity domains of the ITP Patient Assessment |
| Endpoints: | Questionnaire (ITP-PAQ) in adult participants (≥18 years) |
| Quality of Life | Change from baseline in disease-specific QOL as measured by |
| (QOL) | the Kids' ITP Tools (ITP-KIT) score in pediatric participants |
| Endpoints | |
| Exploratory | Proportion of participants able to achieve platelet |
| Endpoints (24- | counts ≥50 × 10⁹/L for 4 out of last 8 weeks of the 24-week |
| wk Blinded | treatment period |
| Period) | (1) Percentage of weeks with platelet count of ≥50 × 10⁹/L or (2) |
| | percentage of weeks with platelet counts between ≥30 × 10⁹/L |
| | and <50 × 10⁹/L and at least doubled from baseline over the 24-week |
| | blinded treatment period in the absence of rescue therapy |
| | Proportion of participants with a complete response (defined as |
| | platelet counts of ≥100 × 10⁹/L on 2 consecutive visits ≥5 days |
| | apart and no bleeding or rescue ITP medication use on and |
| | through these two visits) |
| | Proportion of participants with platelet counts of ≥50 × 10⁹/L on 2 |
| | consecutive visits at least 5 days apart with no rescue therapy on |
| | and through those 2 visits |
| | Proportion of participants with platelet counts >250 × 10⁹/L |
| | or >450 × 10⁹/L for patients receiving concomitant TPO-RA |
| | Change from baseline in IBLS assessment at weeks 13 and 25 |
| | Change from baseline and change from week 13 on the |
| | Fatigue/Sleep (item 10 of the ITP-PAQ; physical fatigue), |
| | Psychological Health, Fear, Social Activity, Women's |
| | Reproductive Health, and Work domains of the ITP-PAQ in adult |
| | participants (≥18 years) at week 25 |
| | Change from baseline on the Psychological Health, Fear, Social |
| | Activity, Women's Reproductive Health, and Work domains of |
| | the ITP-PAQ in adult participants (≥18 years) at week 13 |
| | Change from baseline in QOL as measured by the Euroqol-5 |
| | Dimensions-5 Level (Euroqol-5D-5L) in adult participants (≥18 |
| | years) |
| | Change from baseline in disease-related symptom severity as |
| | measured by the Patient Global Impression of Severity (PGIS) |
| | scale |
| | Change from baseline in disease-related fatigue severity as |
| | measured by the Patient Global Impression of [Fatigue] Severity |
| | (PGIS-Fatigue) scale |
| | Patient perception of disease-related symptom improvement as |
| | measured by the Patient Global Impression of Change (PGIC) |
| | scale |
| | PK parameters as assessed by population pharmacokinetic |
| | analysis |
| | BTK occupancy |
| | Changes from baseline in TPO levels, T-lymphocytes/B- |
| | lymphocytes/natural killers (T/B/NK) counts, immunoglobulin |
| | (IgG, IgG1, IgG4, IgM, IgE) levels |
| | (Optional) Vaccine-specific IgG response during treatment |
| Exploratory | Proportion of participants who received the placebo during the |
| Endpoints | blinded part who achieve platelet counts ≥50 × 10⁹/L for two- |
| (Open-label and | thirds of ≥10 available weekly scheduled platelet measurements |
| LTE Period) | during the last 16 of 28 weeks in the open-label period in the |
| | absence of rescue medication, provided that at least 3 available |
| | weekly scheduled platelet measurements were ≥50 × 10⁹/L during |
| | the last 8 weeks of the 28-week open-label period |
| | Percentage of weeks with (1) platelet counts of ≥50 × 10⁹/L or (2) |
| | platelet counts between ≥30 × 10⁹/L and <50 × 10⁹/L and at least |
| | doubled from baseline |
| | Proportion of participants with a complete response (platelet |
| | count ≥100 × 10⁹/L on 2 consecutive visits ≥5 days apart and no |
| | bleeding or rescue ITP therapy use on and through those two |
| | visits |
| | Proportion of participants with platelet counts >250 × 10⁹/L |
| | or >450 × 10⁹/L for patients receiving concomitant TPO-RA |
| | Proportion of patients requiring rescue medication |
| | Change from baseline on the Fatigue/Sleep (Item 10 of the ITP- |
| | PAQ; physical fatigue), Psychological Health, Fear, Social |
| | Activity, Women's Reproductive Health, and Work domains of |
| | the ITP-PAQ in adult participants (≥18 years) |
| | Changes from baseline in IBLS assessment |
| | Change from baseline on the Psychological Health, Fear, Social |
| | Activity, Women's Reproductive Health, and Work domains of |
| | the ITP-PAQ in adult participants (≥18 years) |
| | Change from baseline in QOL as measured by the EuroQOL- |
| | Dimensions-5 Level in adult participants (≥18 years) |
| | Changes from baseline on CS or TPO-RA dose |
| | Proportion of patients who switch to rilzabrutinib monotherapy |
| | during the first year of the LTE period |
| | Proportion of participants who decrease their CS dose >50% |
| | relative to baseline values during the first year of the LTE |
| | Proportion of participants who manage to reduce their dose or |
| | stop TPO-RA agonists during the first year of the LTE |
| | Change from baseline in IBLS assessment at Week 25 (EU and |
| | UK only) |
| | Proportion of patients who achieve a stable platelet response (no |
| | 2 scheduled visits, at least 4 weeks apart, with a platelet |
| | count <50 × 10⁹/L, without an intervening visit with a platelet |
| | count ≥50 × 10⁹/L), within a period of 24 weeks following initial |
| | achievement of the platelet response (the initial platelet response |
| | is defined as a platelet count ≥50 × 10⁹/L within 12 weeks of |
| | initiation of treatment) |
| | Frequency and severity of treatment emergent adverse events, |
| | including assessment by physical examination, ECG, clinical |
| | laboratory tests (serum chemistry and hematology, except for |
| | platelet counts included in the primary efficacy endpoint), and |
| | vital signs |
| | Frequency and severity of bleeding treatment emergent adverse |
| | events |
| | Plasma concentration of rilzabrutinib |
| | Change from baseline on the Symptoms, Bother-Physical Health, |
| | and Activity domains of the ITP-PAQ in patients ≥18 years |
| | Change from baseline in disease-specific QOL as measured by |
| | the ITP-KIT in pediatric patients |

Secondary safety endpoints include an evaluation of the frequency and severity of treatment-emergent adverse events and bleeding events. Safety will be assessed by the incidence, severity, and causal relationship of treatment-emergent adverse events, including clinically significant changes in physical examination, vital signs, electrocardiogram, and laboratory parameters. Intensity of adverse events is graded based on the modified Common Terminology Criteria for Adverse Events, version 5.0. Additional endpoints are detailed in Table 2.

Disease-specific assessment tools include Idiopathic Thrombocytopenic Purpura Bleeding Scale (IBLS), a bleeding assessment comprising 11 site-specific grades assessed at 9 anatomical sites by history over the period before the visit; the ITP-PAQ™, a disease-specific instrument designed to measure quality of life of adult patients with ITP; and ITP Kids' ITP Tools (ITP-KIT) assessment, which is based on a battery of 3 disease-specific instruments and a self-report form specifically designed for pediatric patients with ITP (Mathias et al. 2007, Mathias et al. 2016, Page et al. 2007). Generic health-related quality of life will be assessed using the EuroQOL-5 Dimension 5 Level (EQ-5D-5L) questionnaire and the Patient Global Impression of Severity Scales (generic, for fatigue, and for change). These assessments will be performed every 4 weeks during the blinded and open-label periods. During the first year of the LTE, they will be performed every 28 days; thereafter, they will be performed every 3 months.

### Statistical Considerations

All randomized patients will be included in the intent-to-treat population and all randomized patients who are exposed to ≥1 dose of study medication will be included in the safety population. Assessment of the primary response endpoint between the 2 arms will be performed using the Cochran-Mantel-Haenszel test. The adult sample size of approximately 194 was selected to achieve >85% power to detect a 20% difference in response rate (e.g., 25% in the rilzabrutinib arm vs. 5% in the placebo arm) with approximately 129 adult patients (≥18 years) in the rilzabrutinib arm and 65 in the placebo arm. The pediatric sample size of 30 patients (20 on rilzabrutinib and 10 on placebo) was determined based on clinical practice and development experiences to adequately describe the safety and efficacy in these patients with ITP.

The data monitoring, collection, audit, and management plans are detailed in the study protocol to ensure data quality. To ensure patient safety, study investigators will proactively follow each patient and promptly notify the study sponsor of adverse events and an independent data and safety monitoring board will regularly review and evaluate unblinded patient safety data. No formal interim analysis is planned for the double-blinded part of the study; final analysis will be completed at the end of the study. Additional analyses with the open-label and long-term extension parts may be performed at the study sponsor's discretion.

### Example 2: Basic Characteristics of Adult Patients with Previously Treated Immune Thrombocytopenia Enrolled in LUNA 3 Phase 3 Placebo-Controlled Study of Rilzabrutinib

The purpose of this study was to examine the basic patient characteristics of adult patients in the LUNA 3 study.

Eligible patients had primary ITP for a duration of >3 months for adults (age ≥18 years) and >6 months for adolescent (age 12-17 years) and pediatric patients (age 10-12 years; EU only), and two average platelet counts <30×10⁹/μL within 2 weeks before treatment (Kuter et al. 2023). Patients were to have had a past response (platelet count ≥50×10⁹/μL) to corticosteroids (CS) or intravenous immunoglobulin (IVIg)/anti-D that was not sustained, or a documented intolerance or insufficient response to any appropriate course of standard-of-care ITP therapy.

Patients were stratified by splenectomy status (yes/no) and thrombocytopenia severity (platelet counts <15×10⁹/μL or ≥15×10⁹/μL), then randomized 2:1 to oral rilzabrutinib 400 mg BID or placebo for up to 24 weeks (the double-blinded period) followed by open-label treatment for 28 weeks, and then a 4-week safety follow-up or, if eligible, continue to a long-term extension. After the first 12 weeks, non-responders could directly join the open-label part or discontinue from the study.

Concomitant stable baseline doses of CS and/or thrombopoietin-receptor agonists (TPO-RAs) were permitted. Patients who received rescue medication were allowed to continue study treatment. The primary endpoint was a durable platelet response, defined as achieving platelet counts ≥50×10⁹/μL for at least two-thirds of ≥8 available weekly measurements during the last 12 weeks (including at least 2≥50×10⁹/μL within the last 6 weeks) of the 24-week blinded treatment period in the absence of rescue therapy.

### Results

Overall, 202 adults with a median age of 47 years (range, 18-80) enrolled from 154 study sites as of Sep. 28, 2023 (see Table 3); enrollment is complete for adults and ongoing for pediatrics. Most adults were female (63%), white (62%) or Asian (32%), and not Hispanic/Latino (77%). Geographic regions enrolling patients included Asia/Pacific (35% of patients), West Europe (24% of patients), South America (17% of patients), East Europe (16% of patients), and North America (8% of patients). Patients had ITP for a median duration of 7.7 years, with 75% having a duration >3 years. The median baseline platelet count was 15×10⁹/μL, and 48% of patients had a baseline platelet count <15×10⁹/μL. Overall, patients had a median of 4 (range: 1-17) unique prior ITP therapies, with 73% having ≥3 prior ITP therapies and 2800 prior splenectomy. The most common prior ITP therapies were CS (96%), TPO-RA (66%), and IVIg (530%); patients' prior responses are shown in Table 3.

TABLE 3
LUNA 3 Baseline Patient Demographics, Disease Characteristics,
and Prior Treatment in Adults with ITP.

| | Adult Patients | |
|---|---|---|
| | (N = 202) | |
| Median age (range), years | 47 | (18-80) |
| Age ≥65 years, n (%) | 36 | (18) |
| Sex, n (%) | | |
| Female | 127 | (63) |
| Male | 75 | (37) |
| Geographic region, n (%) | | |
| Asia/Pacific | 70 | (35) |
| West Europe | 49 | (24) |
| South America | 35 | (17) |
| East Europe | 32 | (16) |
| North America | 16 | (8) |
| Median duration of ITP (range), years | 7.7 | (0.3-52.2) |
| Duration of ITP for >3 years, n (%) | 152 | (75) |
| Baseline platelet count,* ×10⁹/L | | |
| Median (range) | 15 | (1-54) |
| Mean (SD) | 15 | (9) |
| Baseline platelet count, n (%) | | |
| <15 × 10⁹/L | 97 | (48) |
| ≥15 × 10⁹/L | 105 | (52) |
| Median number of unique prior ITP | 4 | (1-17) |
| therapies^{†} (range) | | |
| Number of unique prior ITP therapies^{†} | | |
| (n = 201), n (%) | | |
| 1-2 | 54 | (27) |
| 3-4 | 49 | (24) |
| ≥5 | 98 | (49) |
| Prior splenectomy, n (%) | | |
| Yes | 56 | (28) |
| No | 146 | (72) |
| Most common prior ITP medications,^{‡} n (%) | | |
| CS | 193 | (96) |
| TPO-RA | 134 | (66) |
| IVIg | 108 | (53) |
| Immunosuppressants and other immunomodulatory | 106 | (52) |
| agents (including cyclophosphamide) | | |
| Anti-CD20 monoclonal antibody/rituximab | 68 | (34) |
| Fostamatinib | 23 | (11) |
| Anti-D immunoglobulin | 9 | (4) |
| Response to prior ITP medication,^{w} n/n (%) | | |
| CS | 138/193 | (72) |
| TPO-RA | 81/134 | (60) |
| IVIg/anti-D immunoglobulin | 85/111 | (77) |
| Rituximab | 22/68 | (32) |
| Fostamatinib | 7/23 | (30) |

*Baseline, as defined herein.
^{†}Unique prior therapy was identified using standard medication terms. Different corticosteroids counted as 1 therapy. Prior therapies may have included splenectomy.
^{‡}Concomitant stable doses of CS and/or TPO-RA were permitted to continue from baseline during the study.
¹Patient information was unavailable due to lagging trial data entry.
^{w}Response was defined as “resulted in platelet count ≥50 × 10⁹/L” per electronic case report form in patients receiving that medication.

### Example 3: Results of a Phase 3, Multicenter, Randomized, Double-Blind, Placebo-Controlled, Parallel-Group Study with an Open-Label Extension to Evaluate the Efficacy and Safety of Oral Rilzabrutinib (PRN1008) in Adults, Adolescents, and Children with Persistent or Chronic Immune Thrombocytopenia (ITP) (LUNA 3)

Over the course of the 24-week double-blind period, a total of N=202 adults received treatment with 400 mg BID rilzabrutinib (N=133) or a placebo (N=69). The primary endpoint for this study was the percentage of patients achieving a durable response, defined as (1) the achievement of platelet counts of ≥50×10⁹/μL for at least two-thirds of ≥8 available, weekly scheduled platelet measurements during the last 12 weeks of the 24-week blinded treatment period in the absence of rescue therapy (e.g., in 8 of 12 or 6 of 9 counts) and provided that at least 2 available weekly scheduled platelet measurements are at or above 50,000/μL during the last 6 weeks of the 24-week blinded treatment period; or (2) the achievement of platelet counts of ≥50×10⁹/μL for ≥8 out of the 12 scheduled observations in the last 12 weeks of the 24-week blinded treatment period in the absence of rescue medication (EU and UK) (see Table 2). Key secondary endpoints included the number of weeks with platelet counts ≥50×10⁹/μL or number of weeks with platelet counts between ≥30×10⁹/μL and <50×10⁹/μL and at least doubled from baseline during the 24-week blinded treatment period in the absence of rescue medication; the time to first platelet count ≥50×10⁹/μL or time to first platelet count between ≥30×10⁹/μL and <50×10⁹/μL and at least doubled from baseline; the proportion of patients requiring rescue therapy during the 24-week blinded treatment period; change from baseline on ITP-PAQ™ physical fatigue score in adult patients (≥18 years of age) at Week 13 of treatment; and change from baseline in IBLS assessment at Week 25 (EU and UK only) (see Table 2).

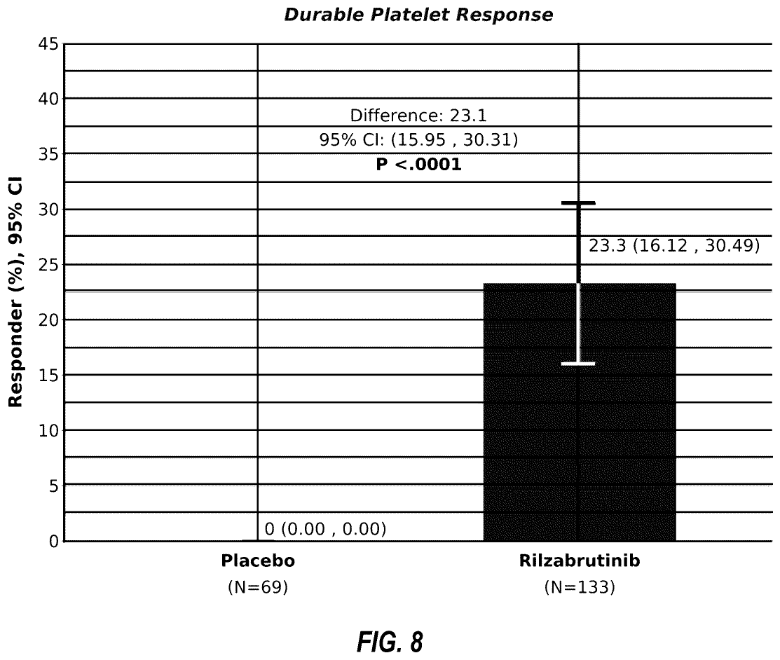

This study met its primary efficacy endpoint. Compared with patients receiving the placebo, patients treated with rilzabrutinib experienced a statistically superior durable platelet response. None of the placebo-treated patients achieved a platelet response; meanwhile, 23% of patients in the rilzabrutinib arm achieved a durable platelet response (p<0.0001; see Table 4 and FIG. 8).

TABLE 4
Summary of the Primary Endpoint.

| | Placebo | Rilzabrutinib |
|---|---|---|
| | (N = 69) | (N = 133) |
| Durable response, n | 0 (0) | 31 (23.3) |
| (%) | | |
| Difference (95% CI), | | 23.1 (15.95, 30.31) |
| % | | |
| P-value | | <.0001 |

Rilzabrutinib is superior to placebo in durable platelet response during the 24-week double-blind treatment^{..}.

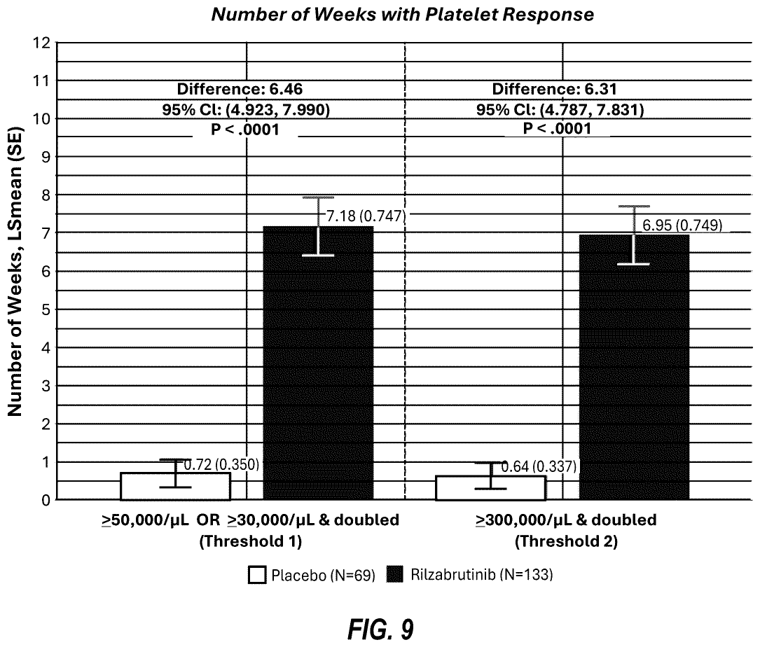

The study also met its key secondary efficacy endpoints. Rilzabrutinib treatment led to a higher number of weeks of platelet response during the double-blinded treatment period, with rilzabrutinib-treated patients experiencing a mean of 7 weeks with platelet counts ≥50×10⁹/μL, as compared to <1 week for placebo-treated patients (p<0.0001)); and a mean of 7 weeks with platelet counts between ≥30×10⁹/μL and <50×10⁹/μL and at least doubled from baseline, as compared to <1 for placebo-treated patients (p<0.0001; see Table 5 and FIG. 9).

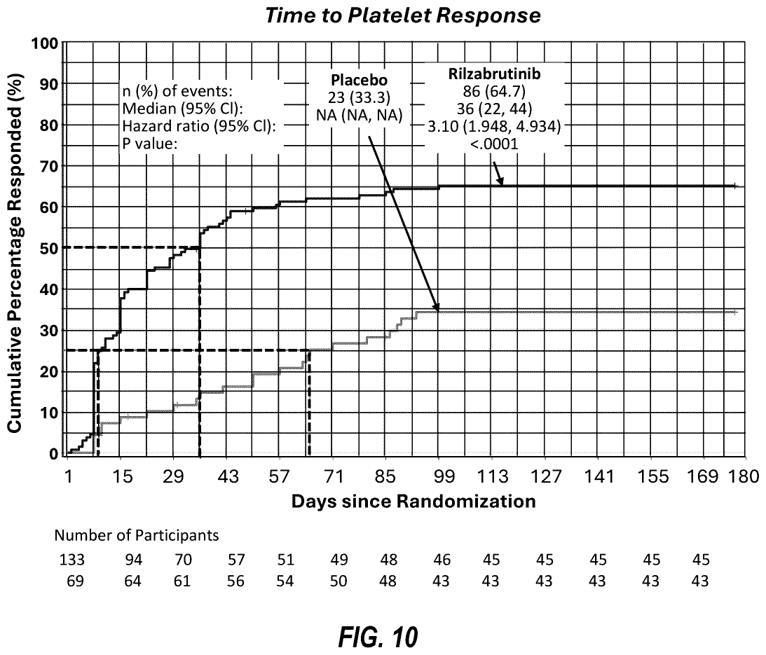

Additionally, the time to first platelet count ≥50×10⁹/μL or time to first platelet count between ≥30×10⁹/μL and <50×10⁹/μL and at least doubled from baseline was significantly shorter in the rilzabrutinib arm than in the placebo arm. In the rilzabrutinib arm, 25% of participants achieved a platelet response by Day 10, while 50% of participants achieved a response by Day 36 (the median time to response). The median was not achieved in the placebo arm. Notably, participants in the rilzabrutinib arm had a 3.1× probability of experiencing a platelet response compared to those in the placebo arm (hazard ratio of 3.1; p<0.0001; see Table 6 and FIG. 10).

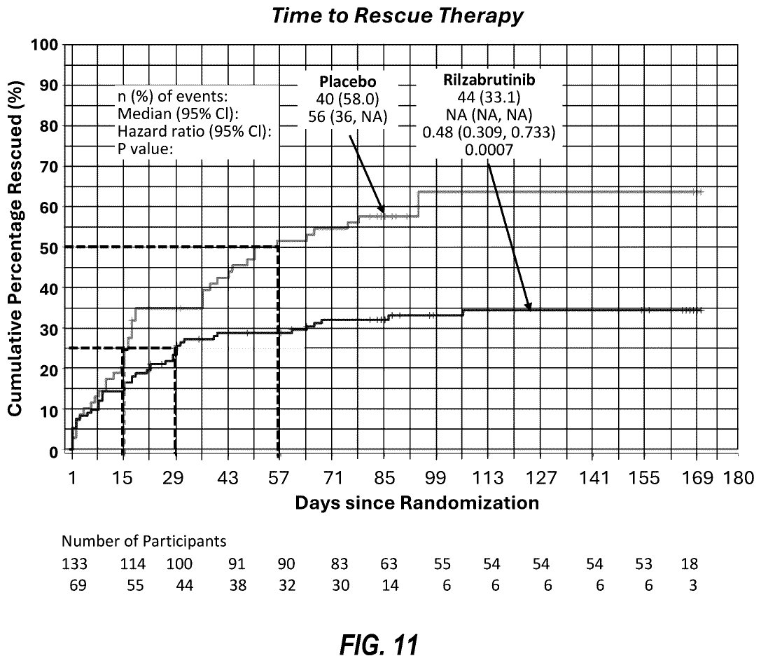

Overall, a lower proportion of participants in the rilzabrutinib arm required rescue therapy over the course of the 24-week double-blinded period; rilzabrutinib was significantly associated with a 52% reduction in the need for rescue therapy (hazard ratio of 0.48; p=0.0007; see Table 7 and FIG. 11). Moreover, the time to rescue therapy was significantly longer in the rilzabrutinib group than the placebo group. By Day 16, 25% of participants in the placebo group had received rescue therapy. While the median time to rescue therapy for the placebo group was Day 56, participants in the rilzabrutinib arm never reached the median.

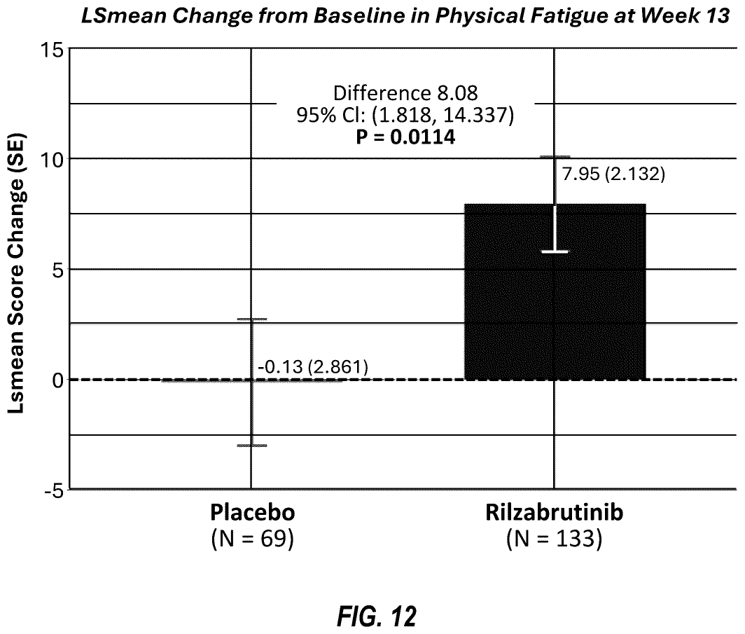

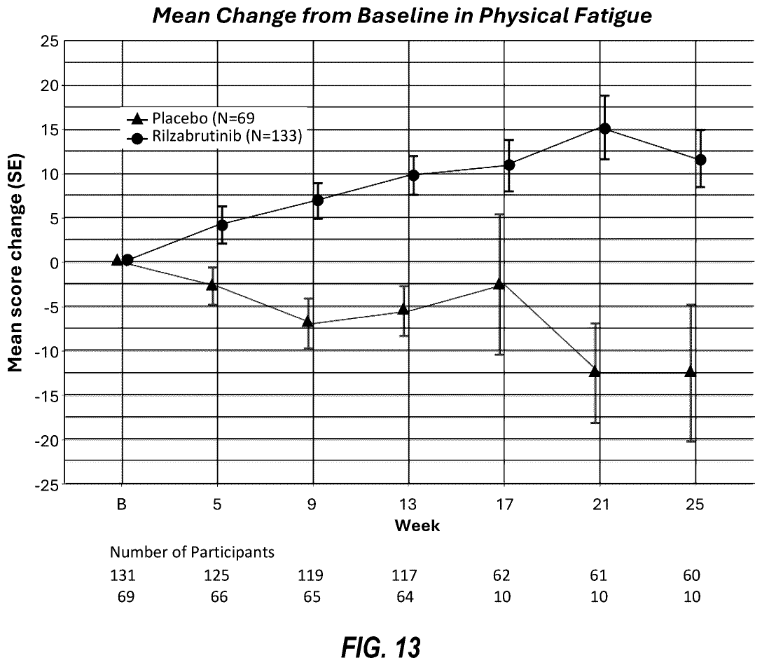

Rilzabrutinib-treated patients also experienced improvements in ITP-PAQ™ physical fatigue score. When compared to patients receiving the placebo, patients receiving rilzabrutinib exhibited a significant improvement in fatigue at week 13 (see Table 8 and FIG. 12). Moreover, a numerically larger treatment effect in fatigue was observed at week 25 as compared to week 13 (FIG. 13).

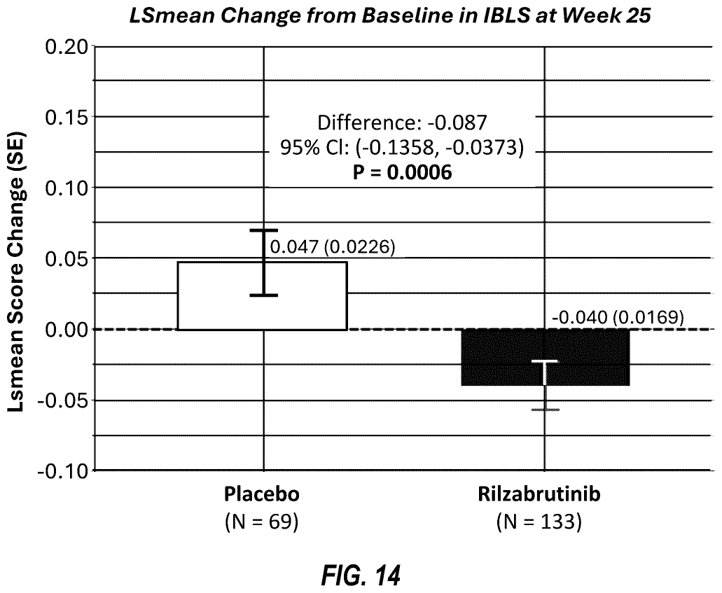

Lastly, rilzabrutinib treatment was associated with a significant reduction in bleeding as assessed by IBLS at week 25. Patients receiving 400 mg BID rilzabrutinib exhibited an LS mean change (SE) from baseline of −0.040 (0.0169), whereas patients receiving the placebo experienced an LS mean change (SE) of 0.047 (0.0226) (p=9.506; see Table 9 and FIG. 14).

TABLE 5
Summary of Key Secondary Endpoint: Number
of Weeks with a Platelet Response.

| | Placebo | Rilzabrutinib |
|---|---|---|
| | (N = 69) | (N = 133) |
| Number of weeks with | | |
| platelet counts 50 × 10⁹/L | | |
| LSmean (SE) | 0.72 (0.350) | 7.18 (0.747) |
| LSmean Difference | | 6.46 (4.923, 7.990) |
| (95% CI) | | |
| P-value | | <.0001 |
| Number of weeks with | | |
| platelet counts | | |
| between ≥30 × 10⁹/L | | |
| and <50 × 10⁹/L | | |
| and at least doubled | | |
| from baseline | | |
| LSmean (SE) | 0.64 (0.337) | 6.95 (0.749) |
| LSmean Difference | | 6.31 (4.787, 7.831) |
| (95% CI) | | |
| P-value | | <.0001 |

TABLE 6
Summary of Key Secondary Endpoint: Time to Platelet Response.

| Time to platelet response | | |
|---|---|---|
| (≥50 k/μL OR ≥30 k/μL & | Placebo | Rilzabrutinib |
| doubled) | (N = 69) | (N = 133) |
| Platelet response, n (%) | 23 (33.3) | 86 (64.7) |
| Median (95% CI), days | NA | 36 (22, 44) |
| Hazard ratio | | 3.1 (1.948, 4.934) |
| P-value | | <.0001 |

TABLE 7
Summary of Key Secondary Endpoint: Proportion
of Patients Requiring Rescue Therapy.

| Time to rescue | Placebo | Rilzabrutinib |
|---|---|---|
| therapy | (N = 69) | (n = 133) |
| Rescued, n (%) | 40 (58) | 44 (33.1) |
| Median (95% CI), | 56 (36, NA) | NA |
| days | | |
| Hazard ratio | | 0.48 (0.309, 0.733) |
| P-value | | 0.0007 |

TABLE 8
Summary of Key Secondary Endpoint: Physical
Fatigue as Measured by ITP-PAO ™.

| Change from baseline | Placebo | Rilzabrutinib |
|---|---|---|
| At Week 13 | (N = 69) | (n = 133) |
| LSmean (SE) | −0.13 (2.861) | 7.95 (2.132) |
| LSmean Difference | | 8.08 (1.818, 14.337) |
| (95% CI) | | |
| P-value | | 0.0114 |
| At Week 25 | | |
| LSmean (SE) | −7.27 (2.989) | 4.69 (2.263) |
| LSmean Difference | | 11.95 (5.416, 18.493) |
| (95% CI) | | |

TABLE 9
Summary of Key Secondary Endpoint: Bleeding
as Assessed by IBLS at Week 25.

| Change from baseline | Placebo | Rilzabrutinib |
|---|---|---|
| At Week 25 | (N = 69) | (N = 133) |
| LSmean (SE) | 0.047 (0.0226) | −0.040 (0.0169) |
| LSmean Difference | | −0.087 (−0.1358, −0.0373) |
| (95% CI) | | |
| P-value | | 0.0006 |

400 mg BID rilzabrutinib was generally well tolerated in adult patients with ITP. A similar percentage of participants in both the placebo and rilzabrutinib arms experienced TEAEs (including TEAEs ≥G3) and SAEs. In the rilzabrutinib arm, the most frequent TEAEs were diarrhea (32.3%), nausea (20.3%), and headache (18%).

### EMBODIMENTS

### Non-Limiting Embodiments of the Disclosure Include:

1. A method for treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has or has been identified as having an initial platelet count of <30,000/μL with no single platelet count >35,000/μL in the 2 weeks prior to the treatment period, and further wherein the human patient has or has been identified as having at least one characteristic chosen from:
   a. a prior response to one or more of IVIg, anti-D, or CSs that was not sustained;
   b. a documented intolerance or insufficient response to any appropriate courses of standard-of-care ITP therapies; and
   c. a contraindication for any appropriate courses of standard-of-care ITP therapies.
2. The method of Embodiment 1, wherein the human patient has or has been identified as having an initial platelet count of <15,000/μL.
3. The method of Embodiment 1, wherein the human patient has or has been identified as having an initial platelet count of ≥15,000/μL.
4. The method of any one of the preceding Embodiments, wherein the human patient achieves a platelet response.
5. The method of any one of the preceding Embodiments, wherein the human patient achieves a durable platelet response.
6. The method of any one of the preceding Embodiments, wherein the human patient achieves a complete platelet response.
7. The method of any one of the preceding Embodiments, wherein the human patient achieves a stable platelet response.
8. The method of any one of the preceding Embodiments, wherein the human patient has at least one platelet count of ≥30,000/μL during the treatment period.
9. The method of any one of the preceding Embodiments, wherein the human patient has at least one platelet count of ≥30,000/μL and at least a doubling of the baseline platelet count during the treatment period.
10. The method of any one of the preceding Embodiments, wherein the human patient has at least one platelet count of ≥30,000/μL and at least a doubling of the baseline platelet count in the absence of rescue medication during the treatment period.
11. The method of any one of the preceding Embodiments, wherein the human patient has at least one platelet count of ≥30,000/μL and at least a doubling of the baseline platelet count during a 24-week treatment period.
12. The method of any one of the preceding Embodiments, wherein the human patient has at least one platelet count of ≥30,000/μL and at least a doubling of the baseline platelet after a 6-month treatment period.
13. The method of any one of the preceding Embodiments, wherein the human patient has at least one platelet count ≥50,000/μL during the treatment period.
14. The method of any one of the preceding claims, wherein the human patient has at least one platelet count ≥50,000/μL in the absence of rescue medication during the treatment period.
15. The method of any one of the preceding Embodiments, wherein the human patient has at least 2 consecutive platelet count of ≥50,000/μL, wherein the platelet counts are ≥5 days apart, in the absence of rescue medication during the treatment period.
16. The method of any one of the preceding Embodiments, wherein the human patient has platelet counts of ≥50,000/μL for at least two-thirds of at least 8 available weekly platelet counts during the last 12 weeks of a 24-week treatment period, further wherein
   a. at least 2 available weekly platelet counts are ≥50,000/μL during the last 6 weeks of a 24-week treatment period; and
   b. the human patient does not require rescue medication.
17. The method of any one of the preceding Embodiments, wherein the human patient has platelet counts of ≥50,000/μL for at least 8 platelet counts during the last 12 weeks of a 24-week treatment period, further wherein the patient does not require rescue medication.
18. The method of any one of the preceding Embodiments, wherein the human patient has no two consecutive platelet counts ≤50,000/μL, wherein the platelet counts occur at least 4 weeks apart within a period of 24 weeks following at least one platelet count of ≥50,000/μL within 12 weeks of initiating rilzabrutinib treatment.
19. The method of any one of the preceding Embodiments, wherein the human patient has platelet counts of ≥50,000 for 4 out of the last 8 weeks of a 24-week treatment period.
20. The method of any one of the preceding Embodiments, wherein the human patient has platelet counts ≥50,000/μL on ≥4 of 6 biweekly platelet counts between weeks 14 and 24 of a 24-week treatment period.
21. The method of any one of the preceding Embodiments, wherein the human patient has platelet counts of ≥50,000 for two-thirds of ≥10 available weekly platelet counts during the last 16 weeks of a 53-week treatment period, further wherein:
   a. at least 3 available weekly platelet measurements are ≥50,000/μL during the last 8 weeks of the 53-week treatment period; and
   b. the human patient does not require rescue medication.
22. The method of any one of the preceding Embodiments, wherein the human patient has:
   a. at least one platelet count ≥50,000/μL; or
   b. at least one platelet count between ≥30,000/μL and <50,000/μL and at least a doubling of the baseline platelet count during a 24-week treatment period in the absence of rescue medication.
23. The method of any one of the preceding Embodiments, wherein the human patient has at least one platelet count ≥100,000/μL during the treatment period, wherein the at least one platelet count occurs in the absence of bleeding.
24. The method of any one of the preceding Embodiments, wherein the human patient has at least 2 platelet counts of ≥100,000/μL on at least 2 consecutive platelet counts ≥5 days apart in the absence of rescue medication during the treatment period.
25. The method of any one of the preceding Embodiments, wherein the human patient has at least one platelet count of ≥250,000/μL during the treatment period.
26. The method of any one of the preceding Embodiments, wherein the human patient has at least one platelet count of ≥450,000/μL during the treatment period.
27. The method of any one of Embodiments 1-5, wherein the human patient has at least one platelet count ranging from 30,000/μL to 100,000/μL during the treatment period, wherein the at least one platelet count is at least double a baseline platelet count and occurs in the absence of bleeding.
28. The method of any one of Embodiments 1-7 or 27, wherein the human patient has at least one platelet count ranging from ≥30,000/μL to <50,000/μL and at least a doubling of a baseline platelet count during the treatment period.
29. The method of any one of the preceding Embodiments, wherein the human patient receives concomitant treatment with TPO-RAs.
30. The method of any one of the preceding Embodiments, wherein the human patient receives concomitant treatment with at least one TPO-RA selected from rTPO, romiplostim, eltrombopag, and avatrombopag.
31. The method of any one of the preceding Embodiments, wherein the human patient has or has been identified as having a history of response to at least one prior line of therapy, wherein at least one prior therapy is chosen from a splenectomy, rituximab, TPO-RAs, intravenous immunoglobin (IVIG), corticosteroids, anti-D immunoglobulin therapy, and immunosuppressive drugs.
32. The method of any one of the preceding claims, wherein the human patient does not require rescue medication during the treatment period.
33. The method of any one of the preceding Embodiments, wherein the human patient has or has been identified as having had a splenectomy prior to the start of the treatment period.
34. The method of any one of Embodiments 1-33, wherein the human patient has or has been identified as having a history of taking rituximab prior to the start of the treatment period.
35. The method of any one of Embodiments 1-33, wherein the human patient has not received prior treatment with rituximab.
36. The method of any one of the preceding Embodiments, wherein the human patient has or has been identified as having a history of taking at least one TPO-RA prior to the start of the treatment period.
37. The method of any one of the preceding Embodiments, wherein the human patient has or has been identified as having a history of taking at least one TPO-RA selected from rTPO, romiplostim, eltrombopag, and avatrombopag prior to the start of the treatment period.
38. The method of any one of Embodiments 1-35, wherein the human patient has not received prior treatment with one or more TPO-RAs.
39. The method of any one of the preceding Embodiments, wherein the human patient has or has been identified as having a history of taking intravenous immunoglobin (IVIG) prior to the start of the treatment period.
40. The method of any one of the preceding Embodiments, wherein the human patient has or has been identified having a history of taking at least one corticosteroid prior to the start of the treatment period.
41. The method of any one of the preceding Embodiments, wherein the human patient has or has been identified as having a history of taking at least one corticosteroid selected from dexamethasone or oral prednisone/prednisolone prior to the start of the treatment period.
42. The method of any one of the preceding Embodiments, wherein the human patient has or has been identified as having a history of taking anti-D immunoglobulin therapy prior to the start of the treatment period.
43. The method of any one of the preceding Embodiments, wherein the human patient has or has been identified as having a history of taking at least one immunosuppressive drug prior to the start of the treatment period.
44. The method of any one of the preceding Embodiments, wherein the human patient has or has been identified as having a history of taking at least one immunosuppressive drug selected from fostamatinib, mycophenolate mofetil (MMF), and cyclosporine prior to the start of the treatment period.
45. The method of any one of the preceding Embodiments, wherein the human patient has or has been identified as having a response to the prior ITP therapy.
46. The method of any one of the preceding Embodiments, wherein the response to the prior ITP therapy comprised a platelet count of <50,000/μL.
47. The method of any one of the preceding Embodiment, wherein the response to the prior ITP therapy was not sustained.
48. The method of any one of the preceding Embodiments, wherein the patient has or has been identified as having a documented intolerance to standard-of-care ITP therapies.
49. The method of any one of the preceding Embodiments, wherein the patient has or has been identified as having a contraindication for standard-of-care ITP therapies.
50. The method of any one of the preceding Embodiments, wherein the human patient has primary ITP.
51. The method of any one of the preceding Embodiments, wherein the human patient is aged ≥18 years.
52. The method of any one of Embodiments 1-50, wherein the human patient is aged <18 years.
53. The method of any one of the preceding Embodiments, wherein the human patient has or has or has been identified as having had ITP for a duration of >1 year.
54. The method of any one of Embodiments 1-52, wherein the human patient does not have chronic ITP.
55. The method of any one of Embodiments 1-52, wherein the human patient has persistent ITP.
56. The method of any one of Embodiments 1-52 wherein the human patient has chronic ITP.
57. The method of any one of Embodiments 1-52, wherein the human patient has relapsing ITP.
58. The method of any one of Embodiments 1-52, wherein the human patient has refractory ITP.
59. The method of any one of the preceding Embodiments, wherein the treatment period is at least 168 days.
60. The method of any one of the preceding Embodiments, wherein the treatment period is at least 364 days.
61. The method of any one of the preceding Embodiments, wherein the treatment period is at least 392 days.
62. The method of any one of the preceding Embodiments, wherein the treatment period is at least 728 days.
63. The method of any one of the preceding Embodiments, comprising administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day.
64. The method of any one of Embodiments 1-63, wherein the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.
65. The method of any one of Embodiments 1-63, wherein the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.
66. The method of any one of Embodiments 1-63, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.
67. The method of any one of Embodiments 1-60, comprising administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day.
68. The method of any one of Embodiments 1-62 or 67, wherein the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
69. The method of any one of Embodiments 1-62 or 67, wherein the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
70. The method of any one of Embodiments 1-62 or 67, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
71. The method of any one of the preceding Embodiments, wherein the at least one compound is orally administered to the human patient.
72. The method of any one of the preceding Embodiments, wherein the at least one compound is administered to the human patient in the form of at least one tablet.
73. The method of any one of the preceding Embodiments, wherein the at least one compound is administered with water.
74. The method of any one of Embodiments 1-50 or 52-73, wherein the patient is 10 to <18 years of age.
75. The method of any one of Embodiments 1-50 or 52-74, wherein the patient is 12 to <18 years of age.
76. The method of any one of Embodiments 1-50 or 52-74, wherein the patient is 10 to <12 years of age.
77. The method of any one of the preceding Embodiments, wherein the patient experiences a reduction in fatigue.
78. The method of any one of the preceding Embodiments, wherein the human patient experiences a reduction in fatigue as determined using ITP Patient Assessment Questionnaire™ (ITP-PAQ™)
79. The method of any one of Embodiments 1-78, wherein the human patient experiences a reduction in fatigue as determined using the ITP Kids' ITP Tools (ITP-KIT).
80. The method of any one of the preceding Embodiments, wherein the human patient experiences a change from baseline in IBLS at weeks 13 and 15 of a treatment period.
81. The method of any one of the preceding Embodiments, wherein the human patient experiences a change from baseline in IBLS at week 25 of a treatment period.
82. The method of any one of the preceding Embodiments, wherein the human patient experiences an improvement in quality of life.
83. The method of any one of the preceding Embodiments, wherein the human patient experiences an improvement in quality of life as determined by the ITP-KIT.
84. The method of any one of the preceding Embodiments, wherein the human patient experiences a change from baseline on the Symptoms, Bother-Physical Health, Activity, Fatigue/Sleep, Psychological Health, Fear, Social Activity, Women's Reproductive Health, and Work domains of the ITP-PAQ™
85. The method of any one of the preceding claims, wherein the human patient experiences less frequent or less severe bleeding than a human patient administered a therapeutically effective amount of another BTK inhibitor
86. The method of Embodiment 1, wherein the human patient has or has been identified as having become refractory to at least one prior line of therapy, for example at least two, for example at least three, for example at least four, for example at least five, for example at least six, for example at least seven, for example at least eight, for example at least nine, for example at least 10, for example at least 11, for example at least 12, for example at least 13, for example at least 14, for example at least 15, for example at least 16, for example at least 17 prior lines of therapy.
87. A method of treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, twice a day for a treatment period, wherein the human patient has or has been identified as having an insufficient response or intolerance to one or more previous treatments.
88. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for treating immune thrombocytopenia (ITP) in a human patient with persistent or chronic ITP in need thereof.
89. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for use as a medicament for treating immune thrombocytopenia (ITP) in a human patient with persistent or chronic ITP in need thereof.
90. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, for use for treating immune thrombocytopenia (ITP) in a human patient with persistent or chronic ITP in need thereof.
91. The use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, as a medicament for treating immune thrombocytopenia (ITP) in a human patient with persistent or chronic ITP in need thereof.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

1-90. (canceled)

91. A method of treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof, twice a day for a treatment period, wherein the human patient has an insufficient response or intolerance to one or more previous treatments, optionally wherein the human patient experiences a change from baseline on the Symptoms, Bother-Physical Health, Activity, Fatigue/Sleep, Psychological Health, Fear, Social Activity, Women's Reproductive Health, and Work domains of the ITP-PAQ™.

92. A method for treating immune thrombocytopenia (ITP) in a human patient with persistent or chronic ITP in need thereof comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has an initial platelet count of <30,000/μL with no single platelet count >35,000/μL in the 2 weeks prior to the treatment period, and further wherein the human patient has at least one characteristic chosen from:
a. a prior response to one or more of IVIg, anti-D, or CSs that was not sustained;
b. a documented intolerance or insufficient response to any appropriate courses of standard-of-care ITP therapies; and
a contraindication for any appropriate courses of standard-of-care ITP therapies

93. The method of claim 92, wherein the human patient achieves at least one of:
a. at least one platelet count of ≥30,000/μL during the treatment period, optionally wherein the human patient has at least a doubling of the baseline platelet count during the treatment period, further optionally wherein the patient does not require rescue medication during the treatment period;
b. at least one platelet count of ≥30,000/μL and at least a doubling of the baseline platelet count during (i) a 24-week treatment period or (ii) a 6-month treatment period;
c. at least one platelet count ≥50,000/μL during the treatment period, optionally wherein the platelet counts are ≥5 days apart, further optionally wherein the human patient does not require rescue medication during the treatment period;
d. platelet counts of ≥50,000 for 4 out of the last 8 weeks of a 24-week treatment period;
e. platelet counts ≥50,000/μL on ≥4 of 6 biweekly platelet counts between weeks 14 and 24 of a 24-week treatment period;
f. at least one platelet count ≥100,000/μL during the treatment period, optionally at least two platelet counts of ≥100,000/μL on at least 2 consecutive platelet counts ≥5 days apart, further optionally wherein the at least one platelet count occurs in the absence of bleeding;
g. at least one platelet count of ≥250,000/μL, optionally ≥450,000/μL, during the treatment period;
h. at least one platelet count ranging from 30,000/μL to 100,000/μL during the treatment period, wherein the at least one platelet count is at least double a baseline platelet count and occurs in the absence of bleeding; or
i. at least one platelet count ranging from ≥30,000/μL to <50,000/μL and at least a doubling of a baseline platelet count during the treatment period.

94. The method of claim 92, wherein the human patient has platelet counts of ≥50,000/μL for at least two-thirds of at least 8 available weekly platelet counts during the last 12 weeks of a 24-week treatment period, further wherein
a. at least 2 available weekly platelet counts are ≥50,000/μL during the last 6 weeks of a 24-week treatment period; and
the human patient does not require rescue medication.

95. The method of claim 92, wherein the human patient has platelet counts of ≥50,000/μL for at least 8 platelet counts during the last 12 weeks of a 24-week treatment period, further wherein the patient does not require rescue medication.

96. The method of claim 92, wherein the human patient has no two consecutive platelet counts ≤50,000/μL, wherein the platelet counts occur at least 4 weeks apart within a period of 24 weeks following at least one platelet count of ≥50,000/μL within 12 weeks of initiating rilzabrutinib treatment.

97. The method of claim 92, wherein the human patient has platelet counts of ≥50,000 for two-thirds of ≥10 available weekly platelet counts during the last 16 weeks of a 53-week treatment period, further wherein:
a. at least 3 available weekly platelet measurements are ≥50,000/μL during the last 8 weeks of the 53-week treatment period; and
b. the human patient does not require rescue medication.

98. The method of claim 92, wherein the human patient has:
a. at least one platelet count ≥50,000/μL; or
b. at least one platelet count between ≥30,000/μL and <50,000/μL and at least a doubling of the baseline platelet count during a 24-week treatment period in the absence of rescue medication.

99. The method of claim 92, wherein the human patient receives concomitant treatment with one or more TPO-RAs, optionally wherein at least one TPO-RA is selected from rTPO, romiplostim, eltrombopag, and avatrombopag.

100. The method of claim 92, wherein the human patient had history of at least one prior line of therapy prior to the start of the treatment period, wherein at least one prior therapy is chosen from a splenectomy, rituximab, TPO-RAs, intravenous immunoglobin (IVIG), corticosteroids, anti-D immunoglobulin, and immunosuppressive drugs, optionally wherein the human patient has a history of response to at least one prior line of therapy.

101. The method of claim 92, wherein the human patient has a history of taking:
a. at least one TPO-RA selected from rTPO, romiplostim, eltrombopag, and avatrombopag;
b. at least one corticosteroid selected from dexamethasone or oral prednisone/prednisolone; or
c. at least one immunosuppressive drug selected from fostamatinib, mycophenolate mofetil (MMF), and cyclosporine;
prior to the start of the treatment period.

102. The method of claim 92, wherein the human patient had a response to the prior ITP therapy, optionally wherein the response to the prior ITP therapy comprised a platelet count of <50,000/μL or wherein the response to the prior ITP therapy was not sustained.

103. The method of claim 92, wherein the patient has a documented intolerance to or contraindication for standard-of-care ITP therapies.

104. The method of claim 92, wherein the human patient has primary ITP.

105. The method of claim 92, wherein the human patient is aged ≥18 years.

106. The method of claim 92, wherein the human patient is aged <18 years.

107. The method of claim 92, wherein the human patient has one or more of:
a. persistent ITP;
b. chronic ITP;
c. relapsing ITP; and
d. refractory ITP.

108. The method of claim 92, wherein the treatment period is at least 168, 364, 392, or 728 days.

109. The method of claim 92, comprising administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day.

110. The method of claim 92, wherein the at least one compound consists of (i) at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof; (ii) at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof; or (iii) a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

111. The method of claim 92, comprising administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day.

112. The method of claim 92, wherein (i) the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile;
(ii) the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; or (iii) the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

113. The method of claim 92, wherein the at least one compound is orally administered to the human patient, optionally in the form of at least one tablet, further optionally with water.

114. The method of claim 92, wherein the patient is 10 to <18 years of age, optionally wherein the patient is (i) 10 to <12 years of age or (ii) 12 to <18 years of age.

115. The method of claim 92, wherein the patient experiences a reduction in fatigue, optionally wherein:
a. the reduction in fatigue is determined using ITP Patient Assessment Questionnaire™ (ITP-PAQ™);
b. the reduction in fatigue is determined using the ITP Kids' ITP Tools (ITP-KIT).

116. The method of claim 92, wherein the human patient experiences a change from baseline in IBLS at (i) weeks 13 and 15 of a treatment period or (ii) week 25 of a treatment period.

117. The method of claim 92, wherein the human patient experiences an improvement in quality of life, optionally wherein the human patient experiences an improvement in quality of life as determined by the ITP-KIT.
